(19) United States
(12) Patent Application Publication
(10) Pub. No.: US 2025/0076286 A1
(43) Pub. Date: Mar. 6, 2025

(54) GENERATION OF NEURAL ORGANIZER ORGANOIDS AND MIDLINE ASSEMBLOIDS FROM HUMAN PLURIPOTENT STEM CELLS

(71) Applicant: The Board of Trustees of the Leland Stanford Junior University, Stanford, CA (US)

(72) Inventors: Sergiu P. Pasca, Menlo Park, CA (US); Neal Dilip Amin, Palo Alto, CA (US); Massimo Mario Onesto, Stanford, CA (US)

(21) Appl. No.: 18/726,382

(22) PCT Filed: Jan. 13, 2023
(86) PCT No.: PCT/US2023/010834

§ 371 Date: Jul. 2, 2024

Related U.S. Application Data
(60) Provisional application No. 63/299,229, filed on Jan. 13, 2022.

(51) Int. Cl. G01N 33/50; C12N 5/079

(52) U.S. Cl. CPC G01N 33/5082; C12N 5/0618; G01N 33/5058; C12N 2501/11; C12N 2501/115; C12N 2501/60; C12N 2506/45; C12N 2509/00; C12N 2513/00

(57) ABSTRACT

Compositions and methods are provided for the in vitro generation of functional human three-dimensional neural organizers that are functionally active and capable of choreographing in vitro midline brain development from human induced pluripotent stem cells (hiPSC). Demonstrated is a model of floor plate organizer ventral midline neurodevelopment, via the expression of a full compendium of axon guidance, morphogen, and cell signaling molecules. Floor plate organoids can be fused with spinal cord organoids into midline assembloids to induce specific cell fate and cell-cell interactions at the interface. This powerful platform can be used to model human neurodevelopment, study human genetic disorders that result from neural development, identify toxic molecules or drugs that disrupt midline brain development, and screen for therapeutics that could repair or rescue these defects.

### CROSS-REFERENCE TO RELATED APPLICATIONS

Pursuant to 35 U.S.C. § 119 (e), this application claims priority to the filing date of U.S. Provisional Patent Application Ser. No. 63/299,229 filed Jan. 13, 2022, the disclosure of which application is herein incorporated by reference.

### BACKGROUND

Neural development is characterized by the generation of a diversity of neuronal subtypes in precise spatial locations. Organizer cell populations serve as powerful neurodevelopmental guideposts that control spatial patterning of neural progenitors and neural connectivity by establishing signaling gradients of morphogens and axon guidance molecules.

The establishment of dorsoventral (DV) identity in the developing neural tube enables the formation of separate progenitor zones and ultimately the generation of distinct neural subtypes. Dorsal forebrain progenitors can produce glutamatergic projection neurons. In contrast, inhibitory GAGAergic interneurons originate from the ganglionic eminences (GE) in the ventral forebrain and migrate dorsally to the cerebral cortex. In addition to establishing the DV axis, the neural tube also undergoes substantial expansion of progenitor populations, a function that ultimately contributes to forebrain size.

When organizer cell type function is disrupted in human neurodevelopment, patients exhibit neuropsychiatric disorders such as holoprosencephaly, complete corpus callosal agenesis or idiopathic psychiatric conditions. However, the biological investigation of human organizers is limited by the lack of access to living neural tissues.

### SUMMARY

Compositions and methods are provided for the in vitro generation of functional human three-dimensional neural organizers that are functionally active and capable of choreographing in vitro midline brain development from human induced pluripotent stem cells (hiPSC). Demonstrated is a model of floor plate organizer ventral midline neurodevelopment, via the expression of a full compendium of axon guidance, morphogen, and cell signaling molecules. Floor plate organoids can be fused with spinal cord organoids into midline assembloids to induce specific cell fate and cell-cell interactions at the interface. This powerful platform can be used to model human neurodevelopment, study human genetic disorders that result from neural development, identify toxic molecules or drugs that disrupt midline brain development, and screen for therapeutics that could repair or rescue these defects.

Compositions and methods are provided for in vitro generation of functional human midline assembloids, which may be generated at least in part from human pluripotent stem cells (hPSCs). Complete assembloids are assembled from component cultured cell systems, where each cultured cell system is designed to provide specific sets of neural and/or organizer cells, and which components are functionally integrated in the assembled organoids. Specifically, ventral midline assembloids are assembled from human floor plate organoids/organoids (hFpO) and human neural tube organoids (hNtO), which may be referred to herein as (hFpO-hNtO). Additionally, dorsal midline assembloids are assembled from human roof plate organoids (hRpO) and hNtO, which may be referred to as hRpO-hNtO. Additionally, dorsal and ventral midline assembloids are assembled from three components such as hRpO-hNtO-hFpO or any other number of combinations of the constituent components. Functionally integrated cells interact in a physiologically relevant manner, e.g. forming inducing ventralization or dorsalization, expression of floor plate and roof plate markers, stimulation of axon extension, secretion of neural chemoattractants or chemorepulsive cues, and the like.

Human spinal organoids (hSpO) are also provided, which can be used, for example, in functional assays of floor plate or roof plate function with midline assembloids.

In some embodiments, one or more such functional assembled organoids, i.e. midline assembloids, are provided, including without limitation a panel of such in vitro derived assembloids, where the panel comprises organoids generated from two or more genetically different cells. In some embodiments the genome of each component organoids: the hFpO component, the hRpO component, the hNtO component, the hSpO component; are the same or different. In some embodiments a panel of such functional assembloids and organoids are subjected to a plurality of candidate agents, or a plurality of doses of a candidate agent. Candidate agents include small molecules, i.e. drugs, genetic constructs that increase or decrease expression of an RNA of interest, infectious agents, electrical changes, and the like. In some embodiments a panel refers to functional assembloids, or a method utilizing patient-specific functional assembloids, from two or more distinct conditions, e.g. different genetic backgrounds, exposure to different drug treatments, exposure to pathogens, etc., and may be three or more, four or more, five or more, six or more, seven or more different conditions.

In some embodiments, methods are provided for determining the activity of a candidate agent on human cells present in the functional organoids or assembloids, i.e. hFpO, hRpO, hNtO, hSpO, the method comprising contacting the candidate agent with one or a panel of functional assembloids and/or organoids. The cells present in the functional assembloids and/or organoids optionally comprise at least one allele encoding a mutation associated with, or potentially associated with, a cortical, spinal or neuromuscular disease; and determining the effect of the agent on morphological, genetic or functional parameters, including without limitation gene expression profiling, migration assays, ventralization, axonal growth and pathfinding assays, atomic force microscopy, super resolution microcopy, light-sheet microscopy, two-photon microscopy, patch clamping, cell death in neurodegenerative disorders, single cell gene expression (RNA-seq), calcium imaging with pharmacological screens, modulation of synaptogenesis and neuromuscular junctions, and the like.

Optionally individual cell types of interest can be isolated from functional assembloids and/or organoids, i.e. hFpO, hRpO, hNtO, hSpO for various purposes. The cells are harvested at an appropriate stage of development, which may be determined based on the expression of markers and phenotypic characteristics of the desired cell type. Cultures may be empirically tested by immunostaining for the presence of the markers of interest, by morphological determination, etc. The cells are optionally enriched before or after the positive selection step by drug selection, panning, density gradient centrifugation, flow cytometry etc. In another embodiment, a negative selection is performed, where the selection is based on expression of one or more of markers found on hESCs or hiPSC, fibroblasts, epithelial cells, and the like. Selection may utilize panning methods, magnetic particle selection, particle sorter selection, fluorescent activated cell sorting (FACS) and the like.

In some embodiments, a method is provided for generation of functional human floor plate organoids (hFpO). In such embodiments, human induced pluripotent cells (hiPS) are aggregated in microwells, and cultured in the presence of one or more SMAD inhibitors, e.g. dorsomorphin and SB-431542. A smoothened agonist, including without limitation SAG, is included in the culture medium after about one day, for a period of from about 4 to about 7 days, e.g. 5 days. Additional factors may include, without limitation, one or more of EGF, FGF2 and RA, and the medium may comprise all of EGF, FGF2 and RA for a period of from about 4 to about 7 days, e.g. 5 days. The resulting floor plate cell population is substantially pure, e.g. from about 50% of the total population, from about 75%, from about 85%, from about 95% or more. The methods may consist or comprise essentially of this culture step. Floorplate cells may be characterized, for example, by expression of the floor plate marker FOXA2. Other markers include expression of morphogen SHH, axon guidance molecule SLIT2, LMX1B, etc.

In some embodiments, a method is provided for generation of functional human roof plate organoids (hRpO). In such embodiments, human induced pluripotent cells (hiPS) are aggregated in microwells, and cultured in the presence of one or more SMAD inhibitors, e.g. dorsomorphin and SB-431542. A BMP agonist, e.g. BMP4 protein or BMP7 protein, is included in the culture medium after about one day, for a period of from about 4 to about 7 days, e.g. 5 days. Additional factors may include, without limitation, one or more of EGF, FGF2 and RA, and the medium may comprise all of EGF, FGF2 and RA for a period of from about 4 to about 7 days, e.g. 5 days. The resulting roof plate cell population is substantially pure, e.g. from about 50% of the total population, from about 75%, from about 85%, from about 95% or more. The methods may consist or comprise essentially of this culture step. Roof plate cells may be characterized, for example, by expression of the roof plate marker LMX1A. Other markers include expression of morphogen BMP4 or BMP7.

In some embodiments, a method is provided for generation of functional neural tube organoids (NtOs). In such embodiments, human induced pluripotent cells (hiPS) are aggregated in microwells, and cultured in the presence of one or more SMAD inhibitors, e.g. dorsomorphin and SB-431542 for a period of 4 to 7 days, e.g. 5 days. The NtOs may then be transferred to a media comprising EGF, FGF2, CHIR, and RA.

In some embodiments, a method is provided for generation of functional human midline assembloids by assembly of the hFpO and/or hRpO with neural organoids such as neural tube organoids, e.g. by dissociating to control for size, and then reaggregating, and joining both organoids in the same well and allowing to fuse.

In some embodiments, a method is provided for generation of a dorsal human spinal organoid (dhSpO)-hFpO-dhSpO midline assembloid by assembly of the hFpO between two dhSpO, e.g. by joining all three organoids in the same well and allowing to fuse. In some embodiments, axons project from one dhSpO through the hFpO to the other dhSpO. In some embodiments, the axons express MAP2.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 Protocol for generation of hFpO and hNtO (may also be referred to as unpatterned) organoids followed by the fusion to create midline assembloids as well as generation of hRpO organoids.

FIG. 2A-2C (A) High efficiency generation of FOXA2⁺ floor plate organizer organoids (hFpO) derived from hiPS cells. (B) Quantification of FOXA2 positive floor plate cells in hNtO and hFpO (C) LMX1A⁺ roof plate cells (green) in roof plate organoids (hRpO) derived from hiPS cells.

FIG. 3 Floor plate organizers express transcription factors, morphogens, and axon guidance molecules.

FIG. 4 Addition of retinoic acid during hFpO induction gives rise to caudal identity.

FIG. 5A-5G Floor plate organizers can induce SHH-dependent ventral patterning in hNtO (A) Schematic representing experimental paradigm for studying hFpO dependent patterning and gene regulation. (B,C,D) Fusion of hNtO with hFpO induces the expression of NKX2.2, FOXA2 and NKX6.1 that increases from 3DAF to 7DAF. (E,F,G) hFpO induced ventralization is completely abolished in the presence of cyclopamine.

FIG. 6A-6F Single-cell transcriptomic characterization of FP-dependent ventral pattern induction and dorsal pattern inhibition. (A) Schematic representing fusion, dissociation, sorting and sequencing paradigm. (B) Separation of cell clusters from control (unfused) and fused hNtOs. (C) Labeling of neural tube regions across cell populations. (D) Modulation of genes associated with activation of SHH pathway. (E) Induction of ventral markers in fused cluster. (F) Repression of dorsal markers in fused cluster.

FIG. 7A-7H Generation of dorsal human spinal organoids and midline crossing assembloids. (A) Protocol for generating dorsal human spinal organoids. (B) UMAP displaying cellular distribution of clusters across three cell lines. (C) Expression of dorsal markers and absence of ventral markers in dhSpOs. (D) Presence of dorsal interneuron neuron populations in dhSpO. (E) Expression of commissural neuron specific genes in dhSpO. (F) Schematic and representative images of midline crossing assembloid generation. (G) IHC images showing FOXA2 expression in the midline and crossing of MAP2 positive commissural fibers. (H) Live-imaging of commissural axons crossing the floor plate in midline assembloids in 21 hour timecourse.

FIG. 8A-8I Human dorsal commissural neurons respond to hFpO secreted proteins in a Robo3-dependent manner. (A) Validation of ROBO3 KO in growth cones (B) and whole organoids (C). (D) Perturbation of netrin-1 induced outgrowth in ROBO3 KO axons. (FG) Increased sensitivity to Slit-2 chemorepulsion in ROBO3 KO. (HI) Reduction in hFpO induced neurite outgrowth in ROBO3 KO organoids in collagen matrix.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds useful for co-administration with the active agents of the invention can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

### Definitions

By “pluripotency” and pluripotent stem cells it is meant that such cells have the ability to differentiate into all types of cells in an organism. The term “induced pluripotent stem cell” encompasses pluripotent cells, that, like embryonic stem cells (hESC), can be cultured over a long period of time while maintaining the ability to differentiate into all types of cells in an organism. hiPSC have a human hESC-like morphology, growing as flat colonies containing cells with large nucleo-cytoplasmic ratios, defined borders and prominent nuclei. In addition, hiPSC express pluripotency markers known by one of ordinary skill in the art, including but not limited to alkaline phosphatase, SSEA3, SSEA4, SOX2, OCT3/4, NANOG, TRA-1-60, TRA-1-81, etc. In addition, the hiPSC are capable of forming teratomas and are capable of forming or contributing to ectoderm, mesoderm, or endoderm tissues in a living organism.

Pluripotent stem cells may be obtained from patient or carrier cell samples, e.g. adipocytes, fibroblasts, keratinocytes, blood cells and the like. Various somatic cells find use as a source of hiPSCs; of particular interest are adipose-derived stem cells, fibroblasts, and the like. The use of hiPSCs from individuals of varying genotypes, particularly genotypes potentially associated with neurologic and neuromuscular disorders are of particular interest.

As used herein, “reprogramming factors” refers to one or more, i.e. a cocktail, of biologically active factors that act on a cell, thereby reprogramming a cell to multipotency or to pluripotency. Reprogramming factors may be provided to the cells, e.g. cells from an individual with a family history or genetic make-up of interest for heart disease such as fibroblasts, adipocytes, etc.; individually or as a single composition, that is, as a premixed composition, of reprogramming factors. The factors may be provided at the same molar ratio or at different molar ratios. The factors may be provided once or multiple times in the course of culturing the cells of the subject invention. In some embodiments the reprogramming factor is a transcription factor, including without limitation, OCT3/4; SOX2; KLF4; c-MYC; NANOG; and LIN-28.

Somatic cells are contacted with reprogramming factors, as defined above, in a combination and quantity sufficient to reprogram the cell to pluripotency. Reprogramming factors may be provided to the somatic cells individually or as a single composition, that is, as a premixed composition, of reprogramming factors. In some embodiments the reprogramming factors are provided as a plurality of coding sequences on a vector. The somatic cells may be fibroblasts, adipocytes, stromal cells, and the like, as known in the art. Somatic cells or hiPSC can be obtained from cell banks, from normal donors, from individuals having a neurologic or psychiatric disease of interest, etc.

Following induction of pluripotency, hiPSC are cultured according to any convenient method, e.g. on irradiated feeder cells and commercially available medium. The hiPSC can be dissociated from feeders by digesting with protease, e.g. dispase, preferably at a concentration and for a period of time sufficient to detach intact colonies of pluripotent stem cells from the layer of feeders. The organoids (also sometimes referred to as spheroids) can also be generated from hiPSC grown in feeder-free conditions, by dissociation into a single cell suspension and aggregation using various approaches, including centrifugation in plates, etc.

Genes may be introduced into the somatic cells or the hiPSC derived therefrom for a variety of purposes, e.g. to replace genes having a loss of function mutation, provide marker genes, etc. Alternatively, vectors are introduced that express antisense mRNA, siRNA, ribozymes, etc. thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as BCL-2. Various techniques known in the art may be used to introduce nucleic acids into the target cells, e.g. electroporation, calcium precipitated DNA, fusion, transfection, lipofection, infection and the like, as discussed above. The particular manner in which the DNA is introduced is not critical to the practice of the invention.

Disease-associated or disease-causing genotypes can be generated in healthy hiPSC through targeted genetic manipulation (CRISPR/CAS9, etc) or hiPSC can be derived from individual patients that carry a disease-related genotype or are diagnosed with a disease. Moreover, neural and neuromuscular diseases with less defined or without genetic components can be studied within the model system. A particular advantage of this method is the fact that edited hiPSC lines share the same genetic background as their corresponding, non-edited hiPSC lines. This reduces variability associated with line-line differences in genetic background. Conditions of neurodevelopmental and neuropsychiatric disorders and neural diseases that have strong genetic components or are directly caused by genetic or genomic alterations can be modeled with the systems of the invention.

The term “organoid” as used herein refers to a 3-dimensional growth of mammalian cells in culture that retains characteristics of a tissue in vivo, e.g., prolonged tissue expansion with proliferation, multilineage differentiation, recapitulation of cellular and tissue ultrastructure, etc. The terms “organoid” and “spheroid” may be used interchangeably.

Calcium sensors. Neural activity causes rapid changes in intracellular free calcium, which can be used to track the activity of neuronal populations. Art-recognized sensors for this purpose include fluorescent proteins that fluoresce in the presence of changes in calcium concentrations. These proteins can be introduced into cells, e.g. hiPSC, by including the coding sequence on a suitable expression vector, e.g. a viral vector, to genetically modify neurons generated by the methods described herein. GCaMPs are widely used protein calcium sensors, which are comprised of a fluorescent protein, e.g. GFP, the calcium-binding protein calmodulin (CaM), and CaM-interacting M13 peptide, although a variety of other sensors are also available. Many different proteins are available, including, for example, those described in Zhao et al. (2011) Science 333:1888-1891; Mank et al. (2008) Nat. Methods 5 (9): 805-11; Akerboom et al. (2012) J. Neurosci. 32 (40): 13819-40; Chen et al. (2013) Nature 499 (7458): 295-300; etc.; and as described in U.S. Pat. Nos. 8,629,256, 9,518,980 and 9,488,642 and 9,945,844.

Optogenetics integrates optics and genetic engineering to measure and manipulate neurons. Actuators are genetically-encoded tools for light-activated control of proteins; e.g., opsins and optical switches. Opsins are light-gated ion channels or pumps that absorb light at specific wavelengths. Opsins can be targeted and expressed in specific subsets of neurons, allowing precise spatiotemporal control of these neurons by turning on and off the light source. Channel rhodopsins typically allow the fast depolarization of neurons upon exposure to light through direct stimulation of ion channels. Chlamydomonas reinhardtii Channelrhodopsin-1 (ChR1) is excited by blue light and permits nonspecific cation influx into the cell when stimulated. Examples of ChRs from other species include: CsChR (from Chloromonas subdivisa), CoChR (from Chloromonas oogama), and SdChR (from Scherffelia dubia). Synthetic variants have been created, for example ChR2 (H134R), C1V1 (t/t), ChIEF; ChETA, VChR1, Chrimson, ChrimsonR, Chronos, PsChR2, CoChR, CsChR, CheRiff, and the like. Alternatively, ChR variants that inhibit neurons have been created and identified, for example GtACR1 and GtACR2 (from the cryptophyte Guillardia theta), and variants such as iChloC, SwiChRca, Phobos, Aurora. Halorhodopsin, known as NpHR (from Natronomonas pharaoni), causes hyperpolarization of the cell when triggered with yellow light, variants include Halo, eNpHR, eNpHR2.0, eNpHR3.0, Jaws. Archaerhodopsin-3 (Arch) from Halorubrum sodomense is also used to inhibit neurons.

Disease relevance. The effect of genetics, drugs, injury and pathogens on neurons, including ventralization, axon growth and guidance, and the like is of particular interest, where efficacy and toxicity may rest in sophisticated analysis of neuronal projection, migratory and electrical interactions with neurons and floor plate cells, or the ability of neurons to form functional networks. The discrepancy between the number of lead compounds in clinical development and approved drugs may partially be a result of the methods used to generate the leads and highlights the need for new technology to obtain more detailed and physiologically relevant information on cellular processes in normal and diseased states.

Spinal cord. The spinal cord extends caudally from the medulla at the foramen magnum and terminates at the upper lumbar vertebrae, usually between L1 and L2, where it forms the conus medullaris. In the lumbosacral region, nerve roots from lower cord segments descend within the spinal column in a nearly vertical sheaf, forming the cauda equina.

The white matter at the cord's periphery contains ascending and descending tracts of myelinated sensory and motor nerve fibers. The central H-shaped gray matter is composed of cell bodies and nonmyelinated fibers. The anterior (ventral) horns of the “H” contain lower motor neurons, which receive impulses from the motor cortex via the descending corticospinal tracts and, at the local level, from internuncial neurons and afferent fibers from muscle spindles. The axons of the lower motor neurons are the efferent fibers of the spinal nerves. The posterior (dorsal) horns contain sensory fibers that originate in cell bodies in the dorsal root ganglia. The gray matter also contains many internuncial neurons that carry motor, sensory, or reflex impulses from dorsal to ventral nerve roots, from one side of the cord to the other, or from one level of the cord to another.

Spinal cord disorders cause various patterns of deficits depending on which nerve tracts within the cord or which spinal roots outside the cord are damaged. Disorders affecting spinal nerves, but not directly affecting the cord, cause sensory or motor abnormalities or both only in the areas supplied by the affected spinal nerves. Corticospinal tract lesions cause upper motor neuron dysfunction. Acute, severe lesions (e.g., infarction, traumatic lesions) cause spinal shock with flaccid paresis (decreased muscle tone, hyporeflexia, and no extensor plantar responses). After days or weeks, upper motor neuron dysfunction evolves into spastic paresis (increased muscle tone, hyperreflexia, and clonus). Extensor plantar responses and autonomic dysfunction are present. Flaccid paresis that lasts more than a few weeks suggests lower motor neuron dysfunction (e.g., due to Guillain-Barré syndrome).

Active agents and cells can be incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the active agents and/or other compounds can be achieved in various ways, usually by oral administration. The active agents and/or other compounds may be systemic after administration or may be localized by virtue of the formulation, or by the use of an implant that acts to retain the active dose at the site of implantation.

In pharmaceutical dosage forms, the active agents and/or other compounds may be administered in the form of their pharmaceutically acceptable salts, or they may also be used alone or in appropriate association, as well as in combination with other pharmaceutically active compounds. The agents may be combined, as previously described, to provide a cocktail of activities. The following methods and excipients are exemplary and are not to be construed as limiting the invention.

For oral preparations, agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Formulations are typically provided in a unit dosage form, where the term “unit dosage form,” refers to physically discrete units suitable as unitary dosages for human subjects, each unit containing a predetermined quantity of active agent in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the active agents can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

Cells for use in the methods as described herein may be separated from a mixture of cells by techniques that enrich for desired cells, or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Techniques for affinity separation may include magnetic separation, using antibody-coated magnetic beads, affinity chromatography, cytotoxic agents joined to a monoclonal antibody or used in conjunction with a monoclonal antibody, e.g., complement and cytotoxic cells, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above.

The separated cells may be collected in any appropriate medium that maintain the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS).

The collected and optionally enriched cell population may be used immediately, or may be frozen at liquid nitrogen temperatures and stored, being thawed and capable of being reused. The cells will usually be stored in 10% DMSO, 50% FCS, 40% RPMI 1640 medium.

The cells may be infused to the subject in any physiologically acceptable medium by any convenient route of administration, normally intravascularly, although they may also be introduced by other routes, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

### Methods

Compositions and methods are provided for in vitro generation of functional human midline assembloids, which may be generated at least in part from human pluripotent stem cells (hPSCs). Complete assembloids are assembled from component cultured cell systems, where each cultured cell system is designed to provide specific sets of neural and/or organizer cells, and which components are functionally integrated in the assembled organoid. Specifically, the midline assembloids are assembled from human floor plate organoids (hFpO) and/or roof plate organoids (hRpO) and human neural tube organoids (hNtO), which may be referred to herein as (hFpO-hNtO). Functionally integrated cells interact in a physiologically relevant manner, e.g. forming inducing ventralization, expression of floor plate markers, stimulation of axon extension, secretion of neural chemoattractants, and the like.

The methods comprise an initial step of differentiating pluripotent cells, including without limitation induced human pluripotent stem cells (hiPSC), into the component structures of human floor plate cells, e.g. in a floor plate organoid. The cells may be functionally assembled with functional human neural tube cells.

Following the initial differentiation into the component organoids, and assembly of the midline assembloid, optionally the midline assembloid or a hFpO is placed adjacent to a spinal cord assembloid, hSpO in culture under conditions permissive for testing functional properties, e.g. response to chemoattractants, axon growth, axon guidance, etc.

In some embodiments the organoids are differentiated from induced human pluripotent stem cells (hiPSC). In some embodiments the hiPSC are derived from somatic cells obtained from neurologically normal individuals. In other embodiments the hiPSC are derived from somatic cells obtained from an individual comprising at least one allele encoding a mutation associated with a neural disease.

Generation of the component organoids and cells comprised therein utilizes a multi-step process. Initially, hiPSC can be obtained from any convenient source, or can be generated from somatic cells using art-recognized methods. The hiPSC are dissociated from feeders (or if grown in feeder free, aggregated in organoids of specific sizes) and grown in suspension culture in the absence of FGF2, preferably when dissociated as intact colonies. In certain embodiments the culture are feeder layer free, e.g. when grown on vitronectin coated vessels. The culture may further be free on non-human protein components, i.e. xeno-free, where the term has its usual art-recognized definition, for example referring to culture medium that is free of non-human serum.

Human Floor Plate Organoids (hFpO)

A suspension culture of human induced pluripotent stem (hiPSC) cells is dissociated into single cells and dispersed into microwell plates to aggregate into organoids of from about 10⁴ to about 10⁵ cells. An effective dose of one or more SMAD inhibitors is added to the medium, for a period of from about 5 to about 7 days, and may be about 6 days. For example, dorsomorphin (DM) can be added at an effective dose of at least about 0.1 μM, at least about 1 μM, at least about 5 μM, at least about 10 μM, at least about 50 μM, up to about 100 μM concentration. Other useful inhibitors include, without limitation, A 83-01; DMH-1; K 02288; ML 347; SB 505124; etc. SB-431542 can be added at an effective dose of at least about 0.1 μM, at least about 1 μM, at least about 5 μM, at least about 10 μM, at least about 50 μM, up to about 100 μM concentration

Critically, the medium is then supplemented with an SHH pathway agonist, e.g. smoothened agonist (SAG) at a concentration of from about 0 to 1 μM, from about 50 to 150 nM, and may be about 100 nM starting after day 1 and continuing to about day 7. FGF2, and retinoic acid are added to the medium starting on day 1 and CHIR was added to the media beginning on day 2 until day 7.

The present disclosure provides methods for producing substantially pure functional human roof plate cells in vitro. In some embodiments, the method for producing substantially pure functional human floor plate cells in vitro comprises culturing in suspension human induced pluripotent cells in the presence of an effective dose of one or more SMAD inhibitors; adding to the suspension culture after one day and continuing for 6 days, an effective dose of an SHH pathway agonist, optionally in the presence of an effective dose of epidermal growth factor, fibroblast growth factor and/or retinoic acid; supplementing the medium with an effective dose of a GSK-3 inhibitor after day 2 and continuing for 6 days; to generate organoids comprising a substantially pure population of human floor plate cells (hFpO).

In some embodiments, the one or more SMAD inhibitor is dorsomorphin or LDN 193189. In a preferred embodiment, the one or more SMAD inhibitor is dorsomorphin and or LDN 193189, and SB-431542.

The hFpOs produced by the methods disclosed herein possess particular characteristics. The particular characteristics include, without limitation, being substantially pure (e.g. are comprised of at least about 80% floor plate cells), express sonic hedgehog (SHH) and SLIT2, secrete factors associated with axon guidance, express FOXA2, express transcription factors, attract or repel neurons from a spinal cord organoid, comprise at least one allele associated with a neuropsychiatric disorder, induce ventralization of neural cells or neural tissues (e.g. neural tube cells or neural tube organoids), etc. In some embodiments, the hFpOs are comprised of a substantially pure population of human floor plate cells. A substantially pure population of human floor plate cells may be comprised of any number of human floor plate cells that is a majority of the population of the cells in the hFpO. For instance, the substantially pure population of human floor plate cells may be comprised of at least about 80%, 85%, 90%, 95%, 99% or greater than at least about 99% human floor plate cells. In some embodiments, the human floor plate cells express FOXA2. In a preferred embodiments, the substantially pure population of human floor plate cells is comprised of at least about 90% FOXA2 expressing cells.

The factors associated with axon guidance may be any factor that has an effect on axon projections. Non-limiting examples of factors that are associated with axon guidance are netrins, semaphorins, and ephrins. Factors associated with axon guidance are well known in the art and have been described by, for example, Lee et al. (Exp Neurobiol. 2019 June; 28 (3): 311-319) which is specifically incorporated by reference herein.

The induction of ventralization in neural cells or tissues results in the alteration of the expression of specific genes. In some embodiments, the ventralization of neural cells or tissues results in an increase in the expression of one or more genes selected from the group consisting of: FOXA2, NKX2-2, OLIG2, NKX6-2 and any combination thereof. In some embodiments, the ventralization of neural cells or tissues results in in a decrease in the expression of one or more genes selected from the group consisting of: PAX6, IRX3, PAX3, PAX7 and any combination thereof. In some embodiments, the ventralization of neural cells or tissues results in increase expression of PTCH1, increase expression of GLI1, decreased expression of GLI3, decrease expression of BOC or any combination thereof. When the hFpO induce ventralization of neural cells or tissues results in increased or decreased expression of a gene, the increase or decrease in expression is an increase or decrease of expression of a gene of the neural cell or tissue relative to the neural cell or tissue in the absence of the hFpO. In a preferred embodiment, the neural cell or tissue is a neural tube cell or neural tube organoid.

In some embodiments, the hFpO comprises at least one allele associated with a neuropsychiatric disorder. The neuropsychiatric disorder may be any neuropsychiatric disorder deemed of interest. Neuropsychiatric disorders that find use in the present disclosure include, with limitation, Timothy syndrome, tuberous sclerosis, 22q11.2 deletion syndrome (also known as DiGeorge syndrome), Autism spectrum disorder, Epilepsy, Schizophrenia, Huntington's disease, Parkinson's disease, Tourette's syndrome, etc.

Human Roof Plate Organoids (hRpO)

A suspension culture of human induced pluripotent stem (hiPSC) cells are dissociated into single cells and dispersed into microwell plates to aggregate into organoids of from about 10⁴ to about 10⁵ cells. An effective dose of one or more SMAD inhibitors is added to the medium, for a period of from about 5 to about 7 days, and may be about 6 days. For example, dorsomorphin (DM) can be added at an effective dose of at least about 0.1 μM, at least about 1 μM, at least about 5 μM, at least about 10 μM, at least about 5 μM, up to about 100 μM concentration. Other useful inhibitors include, without limitation, A 83-01; DMH-1; K 02288; ML 347; SB 505124; etc. SB-431542 can be added at an effective dose of at least about 0.1 μM, at least about 1 μM, at least about 5 μM, at least about 10 μM, at least about 50 μM, up to about 100 UM concentration

Critically, the medium is then supplemented with an BMP pathway agonist, e.g. BMP4 at a concentration of from about 0 to 100 ng/uL, from about 50 to 100 ng/uL, and may be about 100 ng/uL starting after day 1 and continuing to about day 7. FGF2, and retinoic acid are added to the medium starting on day 1 and CHIR was added to the media beginning on day 2 until day 7.

The present disclosure provides methods for producing substantially pure functional human roof plate cells in vitro. In some embodiments, the method for producing substantially pure functional human roof plate cells in vitro comprises culturing in suspension human induced pluripotent cells in the presence of an effective dose of one or more SMAD inhibitors; adding to the suspension culture after one day and continuing for 6 days, an effective dose of an BMP pathway agonist, optionally in the presence of an effective dose of epidermal growth factor, fibroblast growth factor and/or retinoic acid; supplementing the medium with an effective dose of a GSK-3 inhibitor after day 2 and continuing for 6 days; to generate organoids comprising a substantially pure population of human roof plate cells.

In some embodiments, the one or more SMAD inhibitor is dorsomorphin or LDN 193189. In a preferred embodiment, the one or more SMAD inhibitor is dorsomorphin and or LDN 193189, and SB-431542. In some embodiments, the GSK-3 inhibitor is CHIR 99021.

The methods disclosed herein produce a substantially pure population of human roof plate cells. A substantially pure population of human roof plate cells may be comprised of any number of human roof plate cells that is a majority of the population of the cells in the hFpO. For instance, the substantially pure population of human roof plate cells may be comprised of at least about 80%, 85%, 90%, 95%, 99% or greater than at least about 99% human roof plate cells.

In some embodiments, the hRpO comprises at least one allele associated with a neuropsychiatric disorder. The neuropsychiatric disorder may be any neuropsychiatric disorder deemed of interest. Neuropsychiatric disorders that find use in the present disclosure include, with limitation, Timothy syndrome, tuberous sclerosis, 22q11.2 deletion syndrome (also known as DiGeorge syndrome), Autism spectrum disorder, Epilepsy, Schizophrenia, Huntington's disease, Parkinson's disease, Tourette's syndrome, etc.

Human Neural Tube Organoids (hNtO)

A suspension culture of hiPSC is induced to a neural fate. hiPSCs are dissociated and grown in suspension. An effective dose of one or more SMAD inhibitors is added to the medium, for a period at least about 2 days, at least about 3 days, at least about 4 days, at least about 5 days, and up to about 10 days, up to about 9 days, up to about 8 days, up to about 7 days, up to about 6 days, up to about 5 days. For example, dorsomorphin (DM) can be added at an effective dose of at least about 0.1 μM, at least about 1 μM, at least about 5 μM, at least about 10 μM, at least about 5 μM, up to about 100 μM concentration. Other useful inhibitors include, without limitation, A 83-01; DMH-1; K 02288; ML 347; SB 505124; etc. SB-431542 can be added at an effective dose of at least about 0.1 μM, at least about 1 μM, at least about 5 μM, at least about 10 μM, at least about 50 μM, up to about 100 μM concentration.

After about 5 days, about 6 days, about 7 days, about 8 days, about 9 days, about 10 days in suspension culture, the floating organoids are moved to neural media to differentiate neural progenitors. The media is supplemented with a GSK-3 inhibitor, e.g. CHIR 99021 at a concentration of from about 1 to 5 μM, about 2 to 25 μM, and may be around about 3 μM in the presence of retinoic acid at a concentration of from about 10 to 1 μM, from about 50 to 150 nM, and may be about 100 nM, FGF2 at a concentration of from about 0 to 50 ng/ml, from about 2.5 to 25 ng/ml and may be about 10 ng/ml; and EGF at a concentration of from about 1 to 50 ng/ml, from about 2.5 to 25 ng/ml and may be about 20 ng/ml, for a period of from about 3 to 7 days, and may be around about 5 days; and retinoic acid at a concentration of from about 10 to 1 μM, from about 50 to 150 nM, and may be about 100 nM.

The present disclosure provides methods for producing a human neural tube organoids (hNtO). In some embodiments, the method for producing a human neural tube organoid comprises (a) culturing in suspension human induced pluripotent cells in the presence of an effective dose of one or more SMAD inhibitors for 4 to 6 days; (b) transferring the cells of step (a) to a suspension culture comprising an effective dose of epidermal growth factor, fibroblast growth factor, a GSK-3 inhibitor, and retinoic acid; (c) culturing n suspension the cells of step (b) for 1 to 5 days; to generate organoids comprising human neural tube cells.

In some embodiments, the one or more SMAD inhibitor is dorsomorphin or LDN 193189. In a preferred embodiment, the one or more SMAD inhibitor is dorsomorphin and or LDN 193189, and SB-431542. In some embodiments, the GSK-3 inhibitor is CHIR 99021.

In some embodiments, the hNtO comprises at least one allele associated with a neuropsychiatric disorder. The neuropsychiatric disorder may be any neuropsychiatric disorder deemed of interest. Neuropsychiatric disorders that find use in the present disclosure include, with limitation, Timothy syndrome, tuberous sclerosis, 22q11.2 deletion syndrome (also known as DiGeorge syndrome), Autism spectrum disorder, Epilepsy, Schizophrenia, Huntington's disease, Parkinson's disease, Tourette's syndrome, etc.

Human Spinal Cord Organoids (hSpO)

To generate spinal cord organoids, hiPSCs are dissociated and grown in suspension; then induced to a neural fate by SMAD inhibitors, e.g. dorsomorphin at a concentration of from about 1 to 50 μM, about 2 to 25 μM, and may be around about 5 μM; and SB-431542 at a concentration of from about 2 to 100 μM, about 5 to 50 μM, and may be around about 10 μM. The cells are cultured in this medium for periods of from about 2 to about 5 days, and may be about 4 days; after which time the medium is supplemented with a GSK-3 inhibitor, e.g. CHIR 99021 at a concentration of from about 1 to 50 μM, about 2 to 25 μM, and may be around about 3 μM in the presence of retinoic acid at a concentration of from about 10 to 1 μM, from about 50 to 150 nM, and may be about 100 nM, FGF2 at a concentration of from about 0 to 50 ng/ml, from about 2.5 to 25 ng/ml and may be about 10 ng/ml; and EGF at a concentration of from about 1 to 50 ng/ml, from about 2.5 to 25 ng/ml and may be about 20 ng/ml, for a period of from about 3 to 7 days, and may be around about 5 days. At about day 11, the medium is then supplemented with an SHH pathway agonist, e.g. smoothened agonist (SAG) at a concentration of from about 0 to 1 μM, from about 50 to 150 nM, and may be about 100 nM. The cells are maintained in the medium for an additional time, up to about day 18.

After about 18 days, the organoids are maintained in culture in neural medium supplemented with BDNF at a concentration of from about 1 to 50 ng/ml, from about 2.5 to 25 ng/ml and may be about 20 ng/ml; IGF at a concentration of from about 1 to 50 ng/ml, from about 2.5 to 25 ng/ml and may be about 10 ng/ml, L-ascorbic acid at a concentration of from about 10 to 500 nM, from about 50 to 250 nM, and may be about 200 nM; and cAMP at a concentration of from about 10 to 500 nM, from about 50 to 150 nM, and may be about 62.5 nM. After such culture, the organoids can be maintained for extended periods of time in neural medium in the absence of growth factors, e.g. for periods of 1, 2, 3, 4, 5, 6, 7, 8, 9, 10, 11, 12, 24, 36 months or longer. Spinal cord organoids are known in the art and have been described in, for example, Anderson et al. (Cell. 2020 Dec. 23; 183 (7): 1913-1929.e26.) and U.S. patent application Ser. No. 17/253,038 each of which are specifically incorporated by reference herein.

In some embodiments, the spinal cord organoid is a dorsal spinal cord organoid. When the spinal cord organoid is a dorsal spinal cord organoid, the spinal cord organoid may be grown in culture conditions lacking factors that ventralize tissue. Factors that ventralize tissue include, without limitation, SAG, FGF2, etc. Dorsal spinal cord organoids of the present disclosure do not express ventral markers. Ventral markers may be any ventral marker gene that is known to be associated with ventralization of spinal cord cells. Ventral markers of interest include, without limitation, FOXA2, NKX2.2, OLIG2, NKX6.2, etc. In some embodiments, the dhSpO comprises dorsal interneurons. In some embodiments, the dhSpO comprises commissural neurons.

In some embodiments, the hSpO or dhSpO comprises at least one allele associated with a neuropsychiatric disorder. The neuropsychiatric disorder may be any neuropsychiatric disorder deemed of interest. Neuropsychiatric disorders that find use in the present disclosure include, with limitation, Timothy syndrome, tuberous sclerosis, 22q11.2 deletion syndrome (also known as DiGeorge syndrome), Autism spectrum disorder, Epilepsy, Schizophrenia, Huntington's disease, Parkinson's disease, Tourette's syndrome, etc.

hFpO-hNtO Midline Assembloid

To generate midline assembloids (hFpO-hNtO midline assembloids), hFpO and hNtO may be generated separately, and later assembled by placing them in close proximity with each other in ultra low attachment round bottom 96 well plates from at least about 12 hours to at least about 48 hours. Preferably the hFpO and hNtO are placed in close proximity for 24 hours. Organoids may be assembled in neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with FGF2, EGF, CHIR, and RA. In some embodiments, the media was changed daily. Assembly may be performed at least about 8 days to 12 days of hFpO and hNtO culture.

The present disclosure provides methods for producing a hFpO-hNtO midline assembloids. In some embodiments, the method for producing a hFpO-hNtO midline assembloid comprises (a) culturing in suspension human induced pluripotent cells in the presence of an effective dose of one or more SMAD inhibitors for 4 to 6 days; (b) transferring the cells of step (a) to a suspension culture comprising an effective dose of epidermal growth factor, fibroblast growth factor, a GSK-3 inhibitor, and retinoic acid; (c) culturing n suspension the cells of step (b) for 1 to 5 days thereby producing a hNtO; and (d) culturing in suspension the hFpO and the hNtO under conditions permissive for cell fusion in a neural medium, such that the hFpO induces ventralization of the hNtO in the hFpO-hNtO midline assembloid.

The hFpO-hNtO midline assemboids disclosed herein have hFpOs that induce ventralization of the hNtO in the hFpO-hNtO midline assembloid. The ventralization of hNtO by hFpO in the hFpO-hNtO midline assembloid may result in a range of different changes in hNtOs. For instance, the ventralization of hNtO may result in a change in the expression of genes in the SHH pathway in the hNtO, an increase in ventral marker expression in hNtO, or a decrease in dorsal marker expression in hNtO. The change in the expression of genes in the SHH pathway may be any gene in the SHH pathway. SHH pathway genes of interest include, without limitation, PTCH1, GLI1, GLI3, BOC, etc., The increase in the expression of ventral markers may be any ventral marker gene. Ventral genes of interest include, without limitation, FOXA2, NKX2-2, OLIG2, NKX6-2, etc. The decrease in the expression of dorsal markers may be any dorsal marker gene. Dorsal genes of interest include, without limitation, PAX6, IRX3, PAX3, PAX7 etc.

In some embodiments, the ventralization of hNtO results in an increase in the expression of one or more genes selected from the group consisting of: FOXA2, NKX2-2, OLIG2, NKX6-2 and any combination thereof. In some embodiments, the ventralization of hNtO results in in a decrease in the expression of one or more genes selected from the group consisting of: PAX6, IRX3, PAX3, PAX7 and any combination thereof. In some embodiments, the ventralization of hNtO results in increase expression of PTCH1, increase expression of GLI1, decreased expression of GLI3, decrease expression of BOC or any combination thereof. When the hFpO inducing ventralization of hNtO results in increased or decreased expression of a gene, the increase or decrease in expression is an increase or decrease of expression of a gene of the hNtO relative to a hNtO in the absence of the hFpO.

hRpO-hNtO Midline Assembloid

To generate midline assembloids (hRpO-hNtO midline assembloids), hRpO and hNtO may be generated separately, and later assembled by placing them in close proximity with each other in ultra low attachment round bottom 96 well plates from at least about 12 hours to at least about 48 hours. Preferably the hRpO and hNtO are placed in close proximity for 24 hours. Organoids may be assembled in neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with FGF2, EGF, CHIR, and RA. In some embodiments, the media was changed daily. Assembly may be performed at least about 8 days to 12 days of hRpO and hNtO culture.

The present disclosure provides methods for producing a hRpO-hNtO midline assembloids. In some embodiments, the method for producing a hFpO-hNtO midline assembloid comprises (a) culturing in suspension human induced pluripotent cells in the presence of an effective dose of one or more SMAD inhibitors for 4 to 6 days; (b) transferring the cells of step (a) to a suspension culture comprising an effective dose of epidermal growth factor, fibroblast growth factor, a GSK-3 inhibitor, and retinoic acid; (c) culturing n suspension the cells of step (b) for 1 day thereby producing a hNtO; and (d) culturing in suspension the hRpO and the hNtO under conditions permissive for cell fusion in a neural medium, such that the hFpO induces dorsalization of the hNtO in the hRpO-hNtO midline assembloid.

dhSpO-hFpO-dhSpO Midline Assembloid

To generate assembloids for studying midline crossing, three day 8 hFpOs may be aligned linearly and in contact on 0.4 μm tissue culture inserts in a 6-well tissue culture plate. From there, neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with EGF, CHIR, FGF2, and RA may be added to the well and incubated overnight to allow for fusion. After 24 hours, two day 35 dhSpOs may be placed in contact with and flanking the linearly fused hFpO. The insert may be then transferred to a new well and neural medium supplemented with brain-derived neurotrophic factor (BDNF; 20 ng/ml, PeproTech, 450-02), IGF, AA, and 5′-cyclic monophosphate sodium salt (cAMP; 100 mM, Millipore Sigma, D0627) may be added to the well. From this point on medium may be changed every other day.

The present disclosure provides methods for producing a dorsal human spinal organoid dhSpO-hFpO-dhSpO midline assembloid. In some embodiments, the method of producing a dhSpO-hFpO-dhSpO midline assembloid comprises (a) culturing in suspension human induced pluripotent cells in the presence of an effective dose of one or more SMAD inhibitors for 4 to 6 days; (b) transferring the cells of step (a) to a suspension culture comprising an effective dose of epidermal growth factor a GSK-3 inhibitor, and retinoic acid; (c) culturing in suspension the cells of step (b) for 10-15 days; (d) culturing in suspension the cells of step (c) in neural media comprising at least one compound selected from the group consisting of brain-derived neurotrophic factor (BDNF), Insulin-like growth factor (IGF), L-Ascorbic Acid 2-phosphate Trisodium Salt (AA), N6, 2′-O-Dibutyryladenosine 3′, 5′-cyclic monophosphate sodium salt (cAMP), thereby generating a dhSpO and (d) culturing the hFpO of claim 1 between two dhSpO under conditions permissive for cell fusion in a neural medium, such that axons project from one dhSpO through the hFpO to the other dhSpO.

dhSpO-hFpO-dhSpO midline assembloids produced by the methods disclosed herein have specific characteristics. Specific characteristics include, without limitation, axonal projections from one dhSpO through the hFpO to the other dhSpO, restriction of expression of FOXA2 to the hFpO, the expression of MAP2 in axonal projections, the lack of ventral marker expression in dhSpO, interaction of hFpO with dhSpO commissural axons, etc.

dhSpO-hFpO-dhSpO midline assembloids of the present disclosure possess axonal projections that originate in one dhSpO, cross through the hFpO and end in the other dhSpO. In some embodiments, the axonal projections express MAP2.

When the dhSpO-hFpO-dhSpO midline assembloid has restriction of expression FOXA2 to the hFpO it is meant that only the human floor plate cells or cells that originated therefrom express FOXA2 and the dorsal human spinal cord cells or cells that originated therefrom do not express FOXA2.

### Screening Assays

In screening assays for the small molecules, the effect of adding a candidate agent to functional assembled organoids, i.e. hFpO-hNtO; and dhSpO-hFpO-dhSpO and including without limitation at the initiation of fusion between the spinal cord, and floor plate organoid components to determine the effect on neuronal projection, migration, axon extension, gene expression, cell death or survival (for neurodegeneration related assays) etc. in culture is tested with one or a panel of cellular environments, where the cellular environment includes one or more of: electrical stimulation including alterations in ionicity, stimulation with a candidate agent of interest, contact with other cells including without limitation roof plate cells, floor plate cells, neural tube cells, spinal cord cells; contact with infectious agents, e.g. rabies virus, polio virus, Zika virus, and the like, and where cells may vary in genotype, in prior exposure to an environment of interest, in the dose of agent that is provided, etc. Usually at least one control is included, for example a negative control and a positive control. Culture of cells is typically performed in a sterile environment, for example, at 37° C. in an incubator containing a humidified 92-95% air/5-8% CO₂ atmosphere. Cell culture may be carried out in nutrient mixtures containing undefined biological fluids such as fetal calf serum, or media which is fully defined and serum free. The effect of the altering of the environment is assessed by monitoring multiple output parameters, including morphological, functional and genetic changes.

Live imaging of cells may be performed and cells modified to express a detectable marker. Calcium sensitive dyes can be used, e.g. Fura-2 calcium imaging; Fluo-4 calcium imaging, Cal-590 calcium imaging, GCaMP6 calcium imaging, voltage imaging using voltage indicators such as voltage-sensitive dyes (e.g. di-4-ANEPPS, di-8-ANEPPS, and RH237) and/or genetically-encoded voltage indicators (e.g. ASAP1, Archer) can be used on the intact organoids, assembled organoids, or on cells isolated therefrom.

Methods of analysis at the single cell level are also of interest, e.g. as described above: live imaging (including confocal or light-sheet microscopy), single cell gene expression or single cell RNA sequencing, calcium imaging, immunocytochemistry, patch-clamping, flow cytometry and the like. Various parameters can be measured to determine the effect of a drug or treatment on the functional assembled organoids or cells derived therefrom.

Parameters are quantifiable components of cells, particularly components that can be accurately measured, desirably in a high throughput system. A parameter can also be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Variability is expected and a range of values for each of the set of test parameters will be obtained using standard statistical methods with a common statistical method used to provide single values.

Parameters of interest include detection of cytoplasmic, cell surface or secreted biomolecules, biopolymers, e.g. polypeptides, polysaccharides, polynucleotides, lipids, etc. Cell surface and secreted molecules are a preferred parameter type as these mediate cell communication and cell effector responses and can be more readily assayed. In one embodiment, parameters include specific epitopes. Epitopes are frequently identified using specific monoclonal antibodies or receptor probes. In some cases the molecular entities comprising the epitope are from two or more substances and comprise a defined structure; examples include combinatorically determined epitopes associated with heterodimeric integrins. A parameter may be detection of a specifically modified protein or oligosaccharide. A parameter may be defined by a specific monoclonal antibody or a ligand or receptor binding determinant.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, New York, (1996), Ninth edition, under the sections: Drugs Acting at Synaptic and Neuroeffector Junctional Sites; Cardiovascular Drugs; Vitamins, Dermatology; and Toxicology, all incorporated herein by reference.

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As used herein, the term “genetic agent” refers to polynucleotides and analogs thereof, which agents are tested in the screening assays of the invention by addition of the genetic agent to a cell. The introduction of the genetic agent results in an alteration of the total genetic composition of the cell. Genetic agents such as DNA can result in an experimentally introduced change in the genome of a cell, generally through the integration of the sequence into a chromosome, for example using CRISPR mediated genomic engineering (see for example Shmakov et al. (2017) Nature Reviews Microbiology 15:169). Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; RNAi, encode a dominant negative mutation, or dominant or constitutively active mutations of native sequences; altered regulatory sequences, etc.

Antisense and RNAi oligonucleotides can be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity, e.g. morpholino oligonucleotide analogs.

Agents are screened for biological activity by adding the agent to at least one and usually a plurality of cells, in one or in a plurality of environmental conditions, e.g. following stimulation with an agonist, following electric or mechanical stimulation, etc. The change in parameter readout in response to the agent is measured, desirably normalized, and the resulting screening results may then be evaluated by comparison to reference screening results, e.g. with cells having other mutations of interest, normal astrocytes, astrocytes derived from other family members, and the like. The reference screening results may include readouts in the presence and absence of different environmental changes, screening results obtained with other agents, which may or may not include known drugs, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of selected parameters, in addition to the functional parameters described above. For measuring the amount of a molecule that is present, a convenient method is to label a molecule with a detectable moiety, which may be fluorescent, luminescent, radioactive, enzymatically active, etc., particularly a molecule specific for binding to the parameter with high affinity fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to fluoresce, e.g. by expressing them as green fluorescent protein chimeras inside cells (for a review see Jones et al. (1999) Trends Biotechnol. 17 (12): 477-81). Thus, antibodies can be genetically modified to provide a fluorescent dye as part of their structure

Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. These techniques utilize specific antibodies as reporter molecules, which are particularly useful due to their high degree of specificity for attaching to a single molecular target. U.S. Pat. No. 4,568,649 describes ligand detection systems, which employ scintillation counting. These techniques are particularly useful for protein or modified protein parameters or epitopes, or carbohydrate determinants. Cell readouts for proteins and other cell determinants can be obtained using fluorescent or otherwise tagged reporter molecules. Cell based ELISA or related non-enzymatic or fluorescence-based methods enable measurement of cell surface parameters and secreted parameters. Capture ELISA and related non-enzymatic methods usually employ two specific antibodies or reporter molecules and are useful for measuring parameters in solution. Flow cytometry methods are useful for measuring cell surface and intracellular parameters, as well as shape change and granularity and for analyses of beads used as antibody- or probe-linked reagents. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these.

Both single cell multiparameter and multicell multiparameter multiplex assays, where input cell types are identified and parameters are read by quantitative imaging and fluorescence and confocal microscopy are used in the art, see Confocal Microscopy Methods and Protocols (Methods in Molecular Biology Vol. 122.) Paddock, Ed., Humana Press, 1998. These methods are described in U.S. Pat. No. 5,989,833 issued Nov. 23, 1999.

Neuronal activity parameters. Of interest for the functionally integrated spheroids/organoids, known as assembloids, screening system are parameters related to the ventralization of the neural cells, axon growth, and axon guidance.

Quantitative readouts of neuronal activity parameters may include baseline measurements in the absence of agents or a pre-defined genetic control condition and test measurements in the presence of a single or multiple agents or a genetic test condition. Furthermore, quantitative readouts of neuronal activity parameters may include long-term recordings and may therefore be used as a function of time (change of parameter value). Readouts may be acquired either spontaneously or in response to or presence of stimulation or perturbation of the complete neuronal network or selected components of the network. The quantitative readouts of neuronal activity parameters may further include a single determined value, the mean or median values of parallel, subsequent or replicate measurements, the variance of the measurements, various normalizations, the cross-correlation between parallel measurements, etc. and every statistic used to a calculate a meaningful and informative factor.

Comprehensive measurements of neuronal activity using electrical or optical recordings of the parameters described herein may include spontaneous activity and activity in response to targeted electrical or optical stimulation, including, for example, ChR2 or other light activatable channels or pumps delivered through lentiviruses, AAVs or pseudo rabies viruses. Furthermore, spontaneous or induced neuronal activity can be measured in the self-assembled functional environment and circuitry of the neural culture or under conditions of selective perturbation or excitation of specific subpopulations of neuronal cells as discussed above.

In the provided assays, comprehensive measurements of neuronal activity can be conducted at different time points along neuronal maturation and usually include a baseline measurement directly before contacting the neural culture with the agents of interest and a subsequent measurement under agent exposure. Moreover, long-term effects of agents on neural maturation and development can be assessed by contacting the immature neural culture at an early time point with agents of interest and acquiring measurements of the same cultures after further maturation at a later time point compared to control cultures without prior agent exposure.

In some embodiments, standard recordings of neuronal activity of mature neural cultures are conducted after about 2 weeks, after about 3 weeks, after about 4 weeks, after about 6 weeks, after about 8 weeks following fusion (i.e. after mixing the different subdomain components of the culture). Recordings of neuronal activity may encompass the measurement of additive, synergistic or opposing effects of agents that are successively applied to the cultures, therefore the duration recording periods can be adjusted according to the specific requirements of the assay. In some embodiments the measurement of neuronal activity is performed for a predetermined concentration of an agent of interest, whereas in other embodiments measurements of neuronal activity can be applied for a range of concentrations of an agent of interest.

In some embodiments the provided assays are used to assess maturation of the neural culture or single component. Maturation of neuronal cells can be measured based on morphology, by optically assessing parameters such as neuromuscular junctions, dendritic arborization, axon elongation, total area of neuronal cell bodies, number of primary processes per neuron, total length of processes per neuron, number of branching points per primary process, and the like.

The results of an assay can be entered into a data processor to provide a dataset. Algorithms are used for the comparison and analysis of data obtained under different conditions. The effect of factors and agents is read out by determining changes in multiple parameters. The data will include the results from assay combinations with the agent(s), and may also include one or more of the control state, the simulated state, and the results from other assay combinations using other agents or performed under other conditions. For rapid and easy comparisons, the results may be presented visually in a graph, and can include numbers, graphs, color representations, etc.

The dataset is prepared from values obtained by measuring parameters in the presence and absence of different cells, e.g. genetically modified cells, cells cultured in the presence of specific factors or agents that affect neuronal function, as well as comparing the presence of the agent of interest and at least one other state, usually the control state, which may include the state without agent or with a different agent. The parameters include functional states such as synapse formation and calcium ions in response to stimulation, whose levels vary in the presence of the factors. Desirably, the results are normalized against a standard, usually a “control value or state,” to provide a normalized data set. Values obtained from test conditions can be normalized by subtracting the unstimulated control values from the test values, and dividing the corrected test value by the corrected stimulated control value. Other methods of normalization can also be used; and the logarithm or other derivative of measured values or ratio of test to stimulated or other control values may be used. Data is normalized to control data on the same cell type under control conditions, but a dataset may comprise normalized data from one, two or multiple cell types and assay conditions.

The dataset can comprise values of the levels of sets of parameters obtained under different assay combinations. Compilations are developed that provide the values for a sufficient number of alternative assay combinations to allow comparison of values.

A database can be compiled from sets of experiments, for example, a database can contain data obtained from a panel of assay combinations, with multiple different environmental changes, where each change can be a series of related compounds, or compounds representing different classes of molecules.

Mathematical systems can be used to compare datasets, and to provide quantitative measures of similarities and differences between them. For example, the datasets can be analyzed by pattern recognition algorithms or clustering methods (e.g. hierarchical or k-means clustering, etc.) that use statistical analysis (correlation coefficients, etc.) to quantify relatedness. These methods can be modified (by weighting, employing classification strategies, etc.) to optimize the ability of a dataset to discriminate different functional effects. For example, individual parameters can be given more or less weight when analyzing the dataset, in order to enhance the discriminatory ability of the analysis. The effect of altering the weights assigned each parameter is assessed, and an iterative process is used to optimize pathway or cellular function discrimination.

The comparison of a dataset obtained from a test compound, and a reference dataset(s) is accomplished by the use of suitable deduction protocols, AI systems, statistical comparisons, etc. Preferably, the dataset is compared with a database of reference data. Similarity to reference data involving known pathway stimuli or inhibitors can provide an initial indication of the cellular pathways targeted or altered by the test stimulus or agent.

A reference database can be compiled. These databases may include reference data from panels that include known agents or combinations of agents that target specific pathways, as well as references from the analysis of cells treated under environmental conditions in which single or multiple environmental conditions or parameters are removed or specifically altered. Reference data may also be generated from panels containing cells with genetic constructs that selectively target or modulate specific cellular pathways. In this way, a database is developed that can reveal the contributions of individual pathways to a complex response.

The effectiveness of pattern search algorithms in classification can involve the optimization of the number of parameters and assay combinations. The disclosed techniques for selection of parameters provide for computational requirements resulting in physiologically relevant outputs. Moreover, these techniques for pre-filtering data sets (or potential data sets) using cell activity and disease-relevant biological information improve the likelihood that the outputs returned from database searches will be relevant to predicting agent mechanisms and in vivo agent effects.

For the development of an expert system for selection and classification of biologically active drug compounds or other interventions, the following procedures are employed. For every reference and test pattern, typically a data matrix is generated, where each point of the data matrix corresponds to a readout from a parameter, where data for each parameter may come from replicate determinations, e.g. multiple individual cells of the same type. As previously described, a data point may be quantitative, semi-quantitative, or qualitative, depending on the nature of the parameter.

The readout may be a mean, average, median or the variance or other statistically or mathematically derived value associated with the measurement. The parameter readout information may be further refined by direct comparison with the corresponding reference readout. The absolute values obtained for each parameter under identical conditions will display a variability that is inherent in live biological systems and also reflects individual cellular variability as well as the variability inherent between individuals.

Classification rules are constructed from sets of training data (i.e. data matrices) obtained from multiple repeated experiments. Classification rules are selected as correctly identifying repeated reference patterns and successfully distinguishing distinct reference patterns. Classification rule-learning algorithms may include decision tree methods, statistical methods, naïve Bayesian algorithms, and the like.

A knowledge database will be of sufficient complexity to permit novel test data to be effectively identified and classified. Several approaches for generating a sufficiently encompassing set of classification patterns, and sufficiently powerful mathematical/statistical methods for discriminating between them can accomplish this.

The data from cells treated with specific drugs known to interact with particular targets or pathways provide a more detailed set of classification readouts. Data generated from cells that are genetically modified using over-expression techniques and anti-sense techniques, permit testing the influence of individual genes on the phenotype.

A preferred knowledge database contains reference data from optimized panels of cells, environments and parameters. For complex environments, data reflecting small variations in the environment may also be included in the knowledge database, e.g. environments where one or more factors or cell types of interest are excluded or included or quantitatively altered in, for example, concentration or time of exposure, etc.

For further elaboration of general techniques useful in the practice of this invention, the practitioner can refer to standard textbooks and reviews in cell biology, tissue culture, embryology, stem cell biology, human development and neurobiology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Generation of Floor Plate and Roof Plate Organoids and Midline Assembloids from Human Pluripotent Stem Cells

Neural development is characterized by the generation of diverse neuronal subtypes in precise spatial locations underlying network connectivity. Organizer cell populations serve as neurodevelopmental guideposts that establish signaling gradients of morphogens and axonal guidance molecules to regulate spatial patterning and later neurodevelopmental stages of synaptic connectivity. When organizer function is disrupted in human development, patients exhibit severe neuropsychiatric disorders such as complete corpus callosal agenesis or holoprosencephaly. However, the biological investigation of human organizers is limited by the lack of access to living neural tissues.

Here, it is reported for the first time the generation from human induced pluripotent stem (hiPSC) cells three-dimensional floor plate organizers that are functionally active and capable of choreographing midline brain development. It was demonstrated that hiPSC cell-derived floor plate recapitulates major stages of ventral midline neurodevelopment via the expression of a full compendium of axon guidance, morphogen, and cell signaling molecules. Moreover, floor plate organoids can be fused with spinal cord organoids into midline assembloids to induce specific cell fate and cell-cell interactions at the interface. hiPSC cell-derived roof plate organoids were similarly generated. This powerful platform can be used to model human neurodevelopment, study human genetic disorders that result from neural development, identify toxic molecules or drugs that disrupt midline brain development, and screen for therapeutics that could repair or rescue these defects.

### Methods

Generation of human floor plate and roof plate organoids (hFpO, hRPO). Human induced pluripotent stem (hiPSC) cells were cultured on vitronectin-coated plates (5 mg ml-1, Thermo Fisher Scientific, A14700) in Essential 8 medium (Thermo Fisher Scientific, A1517001). Cells were passaged every 4 days with UltraPure™ 0.5 mM EDTA, pH 8.0 (Thermo Fisher Scientific, 15575).

For the generation of 3D floor plate organoids, hiPSC cells were incubated with Accutase® (Innovative Cell Technologies, AT104) at 37° C. for 7 min and dissociated into single cells. To obtain uniformly sized organoids, AggreWell-800 (STEMCELL Technologies, 34815) containing 300 microwells was used. Approximately 3×10⁶ single cells were added per AggreWell-800 well in Essential 8 medium supplemented with the ROCK inhibitor Y27632 (10 mM, Selleckchem, S1049), centrifuged at 100 g for 3 min to capture the cells in the microwells, and incubated at 37° C. with 5% CO₂. After 24 h, organoids consisting of approximately 10,000 cells were collected from each microwell by pipetting medium in the well up and down with a cut P1000 pipet tip and transferred into ultra-low attachment plastic dishes (Corning, 3262) in 20% knockout serum medium (KOSR) supplemented with two SMAD pathway inhibitors dorsomorphin (2.5 mM, Sigma-Aldrich, P5499) and SB-431542 (10 mM, R&D Systems, 1614) for the first 5 days.

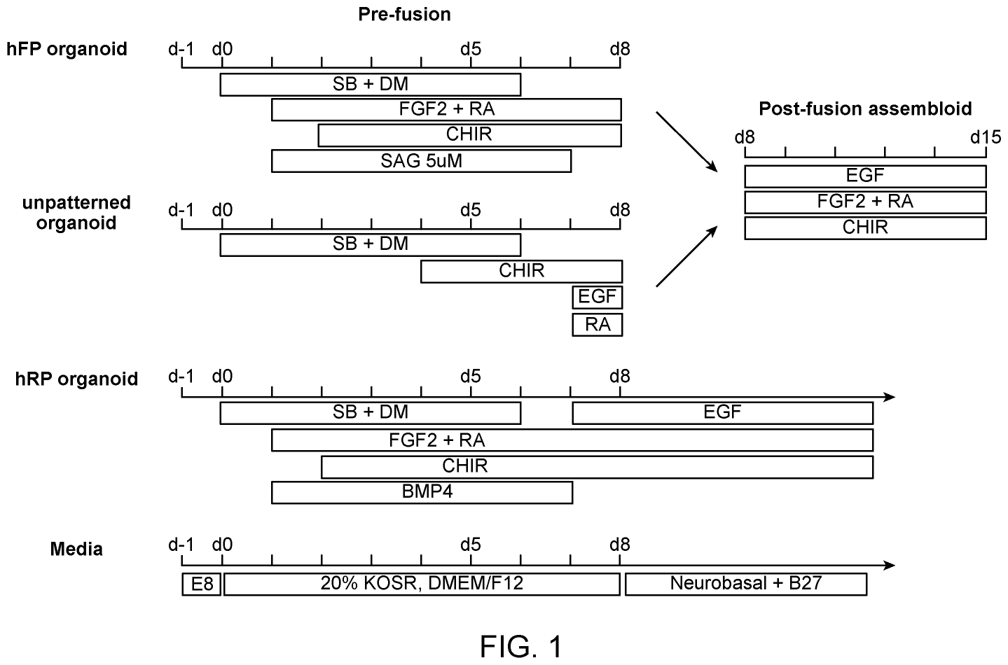

For floor plate induction: SAG, FGF2, and retinoic acid was added to the medium starting on day 1 and CHIR was added to the media beginning on day 2 until day 7. The 20% KOSR medium was changed every day and supplemented with the respective small molecules and growth factors (FIG. 1).

For roof plate induction: BMP4 (human recombinant BMP-4, Preprotech, 100 ng/uL), FGF2, and retinoic acid was added to the medium starting on day 1 and CHIR was added to the media beginning on day 2 until day 7. The 20% KOSR medium was changed every day and supplemented with the respective small molecules and growth factors (FIG. 1).

To prepare floor plate and roof plate organoids for assembly as well as to control for size, the floor plate organoids were dissociated using accutase and seeded at 50,000 cells per well of an ultra low attachment round bottom plate. Cells were seeded in 20% KOSR containing EGF, FGF2, CHIR, retinoic acid and Y27. The plates were centrifuged at 100 G for 3 minutes to collect the dissociated cells at the bottom of the wells. After 24 hours (day 8), the formed hFpOs were pipetted using a wide bore pipette to remove non incorporated cells around the periphery of the aggregate. From here validation of pFpS induction would take place and subsequent applications of this tissue were carried out.

### Single Cell Gene Expression

Dissociated cells were resuspended in ice-cold PBS containing 0.02% BSA and loaded onto a Chromium Single cell 3′ chip (with an estimated recovery of 6,000 cells per channel) to generate gel beads in emulsion (GEMs). scRNA-seq libraires were prepared with the Chromium Single cell 3′ GEM, Library & Gel Bead Kit v3 (10× Genomics, PN: 1000075). Libraries from fused and unfused samples were pooled and sequenced by Admera Health on a NovaSeq S4 (Illumina) using 150×2 chemistry. Demultiplexing, alignment, barcode and UMI counting and aggregation were performed using 10× cloud analysis. Further analysis was performed using the R package Seurat (v3.0) from the Satija Lab (Butler et al., 2018). Cells with more than 10,000 or less than 2,000 detected genes were excluded, as well as those with a mitochondrial content higher than 15%. Genes that were not expressed in at least three cells were excluded. Due to sex differences between hiPS cell lines, all genes on the X and Y chromosomes were removed from the count matrix. Gene expression was then normalized using a global-scaling normalization method (normalization.method=“LogNormalize,” scale.factor=10000, in Seurat), and the 2000 most variable genes were then selected (selection.method=“vst,” in Seurat) and scaled (mean=0 and variance=1, for each gene, as recommended by Seurat) prior to principal component analysis (PCA). The top 30 principal components were utilized to do the clustering with a resolution of 0.5, implemented using the FindNeighbors and FindClusters functions in Seurat.

Real-time qPCR. mRNA was isolated using the RNeasy Mini Kit (Qiagen, 74106) and DNase I, Amplification Grade (Thermo Fisher Scientific, 18068-015), and template cDNA was prepared by reverse transcription using the SuperScript™ III First-Strand Synthesis SuperMix for qRT-PCR (Thermo Fisher Scientific, 11752250). qPCR was performed using SYBR™ Green PCR Master Mix (Thermo Fisher Scientific, 4312704) on a ViiA7 Real-Time PCR System (Thermo Fisher Scientific, 4453545).

Cryoprotection and immunofluorescence staining. hFpO, hNtO and midline assembloids were fixed in 4% paraformaldehyde (PFA)/phosphate buffered saline (PBS) for 1-2 hours on a rocker at 4° C. They were then washed in PBS and transferred to 30% sucrose/PBS overnight until the organoids/assembloids sink in the solution. Subsequently, they were rinsed in optimal cutting temperature (OCT) compound (Tissue-Tek OCT Compound 4583, Sakura Finetek) and embedded. For immunofluorescence staining, 20 μm-thick sections were cut using a Leica Cryostat (Leica, CM1850). Cryosections were blocked in 0.3% Triton X-100 (Millipore Sigma, T9284-100ML), 1% BSA diluted in PBS for 1 h at room temperature. The sections were then incubated overnight at 4° C. with primary antibodies diluted in PBS containing 1% BSA, 0.1% Triton X-100. Samples were then rinsed 3× and incubated for 30 minutes in 1% BSA, 0.1% Triton X-100. Cryosections were then incubated with secondary antibodies in block 1 h. The following primary antibodies were used for staining: anti-FOXA2 (rabbit, Abcam), anti-NKX2.2 (mouse, DSHB, sc-517261), anti-NKX6.1 (mouse, DSHB), LMX1A (rabbit, HPA030088, Atlas antibodies), TFAP2A. Alexa Fluor dyes (Life Technologies) were used at 1:1000 dilution, and nuclei were visualized with Hoechst 33258 (Life Technologies, H3549). Cryosections were mounted for microscopy on glass slides using Aquamount (Polysciences, 18606), and imaged on a Zeiss M1 Axioscope or EVOS microscope. Images were processed in Fiji (NIH).

Production of hFpO Conditioned Medium and Secreted Protein Validation

Conditioned medium of floor plate cells was generated to identify hallmark secreted proteins. A half of a confluent 6-well of hiPSCs was plated on a vitronectin coated 10 cm plate. Typical hiPSC media changes, previously described, were carried out until the cells reached about 50% confluency. From there, floor plate induction was carried out as previously described with respective medias and supplements. On day 7 of induction, the cells were rinsed two times with 10 mL of DPBS (calcium and magnesium free). 10 mL of pre-warmed DMEM: F12 was then added to the plate and incubated overnight (14-20 hrs). The following day conditioned medium was collected and passed through a 0.22 um syringe filter and immediately processed for validation and/or snap frozen and stored at −80 C.

Floor plate secreted proteins were identified via western blot. Protein from conditioned medium was concentrated by adding 25% TCA to 1 mL of sample collected in previous steps and incubated for 30 minutes at 4 C. The solution was then centrifuged at 14,000 rpm for 10 minutes. The supernatant was then removed and washed with acetone. The pellet was then dried at 95 C for 5-10 min and SDS-PAGE was carried out on the protein pellet. The following antibodies were used for western blot: anti-Netrin-1 (Abcam, AF6419), anti-SHH (Invitrogen, 435800), and antiGAPDH (Proteintech, 60004-1-Ig) at 1:10,000 dilution.

Generation of neural tube organoids or organoids (hNtO) and midline assembloids (hNtO-hFpO). To generate hNtO, organoids were generated using the Aggrewell method previously described. The 20% KOSR medium was changed every day and supplemented with dorsomorphin and SB-431542 for 5 Days. On day 6 in suspension, the organoids were transferred to 20% KOSR supplemented with EGF, FGF2, CHIR and retinoic acid. On day 7, hNtO were dissociated and seeded as previously described in ultra-low attachment 96 well plates. To generate midline assembloids (hFpO-hNtO) assembloids, hFpO and hNtO were generated separately, and later assembled by placing them in close proximity with each other in ultra low attachment round bottom 96 well plates for 24 hours. Organoids were assembled in neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with FGF2, EGF, CHIR, and RA. Media was carefully changed daily. Assembly was performed at days (d) 8 of hFpO and hNtO, and they were fixed with 4% PFA/PBS at day 10 and 14 for staining. Cyclopamine was added the same day of fusion and subsequent feeds were also supplemented with this compound throughout the duration of the treatment.

Differentiation of dorsal human spinal organoids (hSpO) and midline assembloids (dhSpO-hFpO-dhSpO) to study crossing of commissural projections. Aggregates of hiPSC cells were generated and collected as previously described in hFpO and hNtO protocols and transferred to poly-HEMA coated 10 cm dishes in Essential 6 medium (Thermo Fisher Scientific, A1516401) supplemented with two SMAD pathway inhibitors-dorsomorphin (2.5 mM, Sigma-Aldrich, P5499) and SB-431542 (10 mM, R&D Systems, 1614). For the first 5 d, Essential 6 medium was changed every day and supplemented with dorsomorphin and SB-431542. To generate hSpO, on day 6 in suspension the organoids were transferred to neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with EGF, CHIR and RA. From day 19, to promote differentiation of the neural progenitors into neurons, the neural medium was supplemented with brain-derived neurotrophic factor (BDNF; 20 ng/ml, PeproTech, 450-02), IGF, AA, and 5′-cyclic monophosphate sodium salt (cAMP; 100 mM, Millipore Sigma, D0627). From this point on medium was changed every other day.

To generate assembloids for studying midline crossing, three day 8 hFpOs were aligned linearly and in contact on 0.4 μm tissue culture inserts in a 6-well tissue culture plate. From there, neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with EGF, CHIR, FGF2, and RA was added to the well and incubated overnight to allow for fusion. After 24 hours, two day 35 dhSpOs were placed in contact with and flanking the linearly fused hFpO. The insert was then transferred to a new well and neural medium supplemented with brain-derived neurotrophic factor (BDNF; 20 ng/ml, PeproTech, 450-02), IGF, AA, and 5′-cyclic monophosphate sodium salt (cAMP; 100 mM, Millipore Sigma, D0627) was added to the well. From this point on medium was changed every other day.

Generation of ROBO3 KO iPSC Line

To validate the functionality of floor plate mechanisms that coordinate midline crossing, =isogenic KO hiPS cell lines targeting ROBO3 (an essential gene for midline crossing) with the CRISPR/Cas9 system were generated. Three sgRNAs targeting an early exon of a specific gene were designed and synthesized by Synthego to induce one deletion. Human iPSCs were dissociated with accutase and 0.5 million cells were mixed with 300 pmol sgRNAs and 40 pmol Cas9 protein (Synthego, SpCas9 2NLS Nuclease (1000 μmol)). Nucleofection was performed using the P3 Primary Cell 4D-NucleofectorTMX Kit L (Lonza, V4XP-3032), a 4D-nucleofector core unit and the X unit (Lonza) (program CA-137). Cells were then seeded onto vitronectin coated 6-well plates in Essential 8 medium supplemented with the ROCK inhibitor Y27632 (10 UM). Essential 8 medium was used for daily medium change. Once confluency was reached 6,250 cells were seeded onto a vitronectin coated 10 cm plate. Single colonies were picked and transferred to a vitronectin coated 96-well plate to generate isogenic lines. Lines were then propagated and deletion of ROBO3 was confirmed via Sanger sequencing, western blot and IHC.

Embedding of organoids in collagen matrix and imaging of hSpO projections to hFpO. A collagen-based solution of 1:2 parts Rat Tail Type I Collagen (BD, 3.83 mg/mL to 1.92 mg/mL), 1:10 10×MEM, and 2:5 NPC medium was prepared over ice and mixed thoroughly. This solution was buffered to a pH between 7-7.4 or until the pH indicator in the MEM solution turned pink using 1M sodium bicarbonate. 40 μL of this solution was pipetted into the bottom of a 48-well glass bottom imaging plate. One day 30 hSpO was immediately positioned about 150 μm from either another day 30 hSpO or day 8 hFpO. The matrix was then left to polymerize at room temperature for 1 hour and subsequently 1 mL of NPC media supplemented with EGF, FGF2, CHIR and RA was added on top of the collagen gels. After 24 hours, the gels were imaged using confocal microscopy.

### Results

Generation of functional human floor plate organoids (hFpO). To develop a model of human organizer regions such as the floor plate which functions to pattern the ventral spinal cord via morphogenetic secretion or cell contact dependent induction, hiPSC cells were dissociated enzymatically into single cells and aggregated them in microwells. After aggregation, these organoids were transferred media supplemented with dual SMAD inhibitors dorsomorphin and SB-431542 for 5 days starting at day 0. Crucial to floor plate induction is the addition of 5 μM smoothened agonist (SAG) on day 1 until day 6. EGF, FGF2 and RA were also added on these days to give rise to a caudal floor plate. The combination of these factors gives rise to a very pure population of cells that have been identified as floor plate using several approaches.

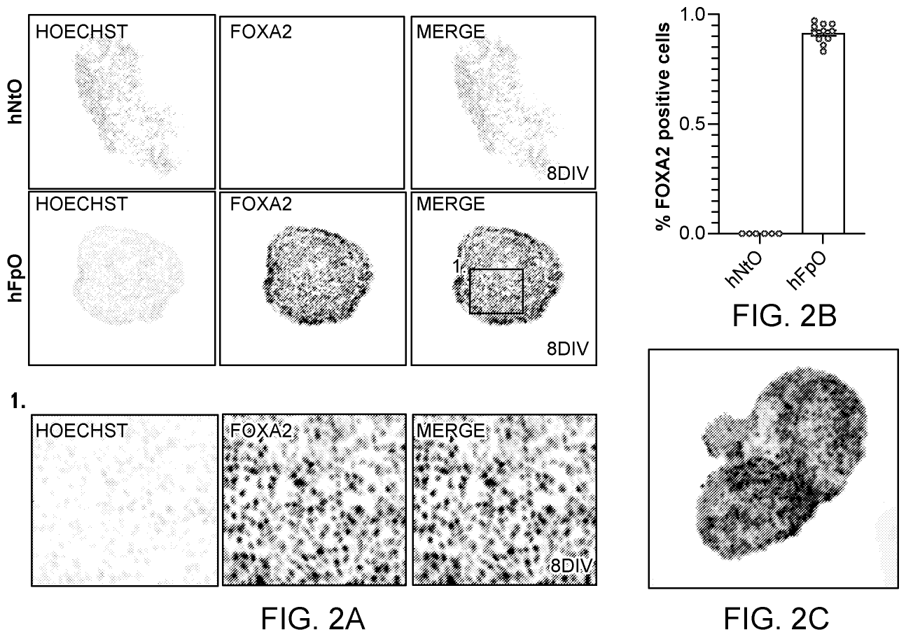

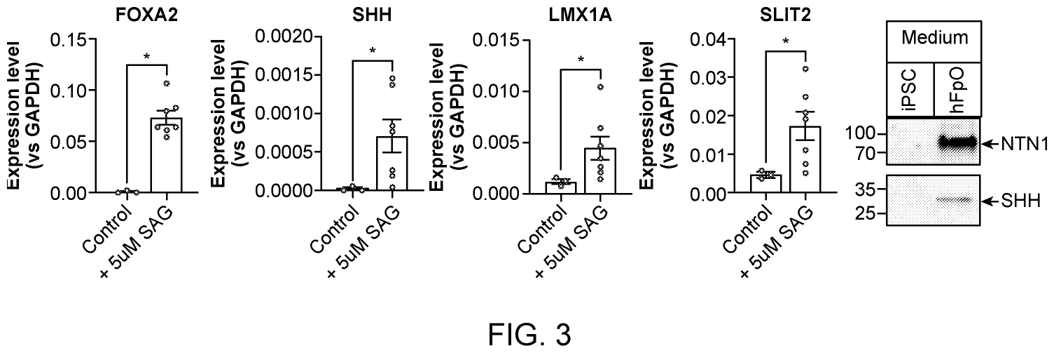

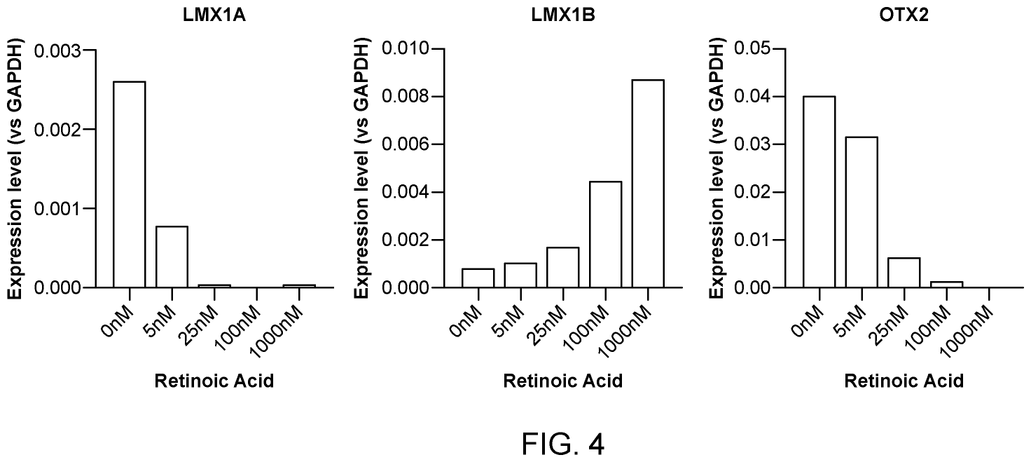

Using immunofluorescence microscopy, the percent of positive floor plate were quantified and ranged from 90-99% using the floor plate marker FOXA2 as an output (FIG. 2). Control organoids that were generated in the absence of SAG showed no induction of FOXA2 positive cells. These groups were also analyzed via RT-qPCR to identify the expression of known secreted factors from the floor plate (FIG. 3). Upon hFpO generation, robust expression of the floor plate marker FOXA2 was identified. The morphogen SHH and the axon guidance molecule SLIT2 was also identified in hFpO. The generation of a caudal floor plate identity was validated and shown by decreasing expression of OTX2 and increasing expression of LMX1B when treated with a series of increasing amounts of retinoic acid (FIG. 4). Taken together, this data demonstrates that organizer regions such as the floor plate can be generated in the form of an organoid.

Generation of functional human roof plate organoids (hRPO). To develop a model of human organizer regions such as the roof plate which functions to pattern the dorsal spinal cord via morphogenetic secretion or cell contact dependent induction, hiPSC cells were disassociated enzymatically into single cells and aggregated them in microwells. After aggregation, these organoids were transferred to media supplemented with dual SMAD inhibitors dorsomorphin and SB-431542 for 5 days starting at day 0. Crucial to floor plate induction is the addition of 100 ng/uL BMP4 on day 1 until day 6. EGF, FGF2 and RA. Using immunofluorescence microscopy, the generation of LMX1A+roof plate cells were identified (FIG. 2). Taken together, this data demonstrates that organizer regions such as the roof plate can be generated in the form of an organoid.

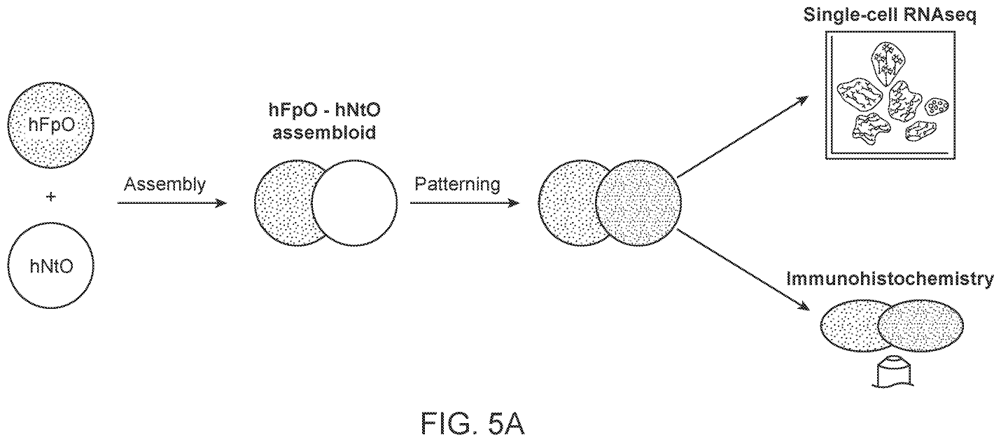

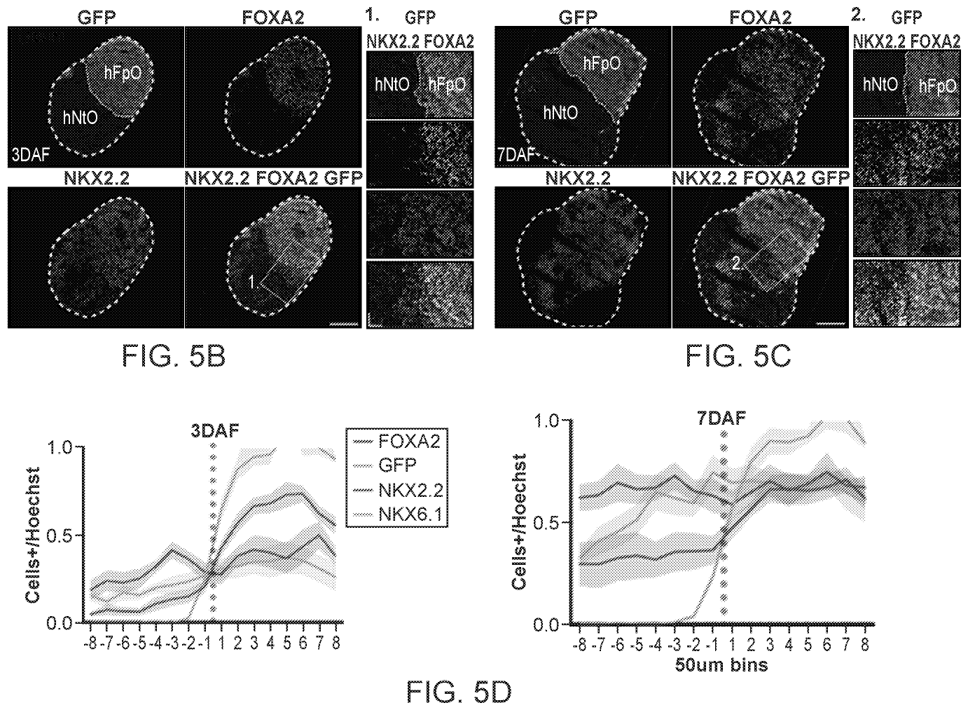

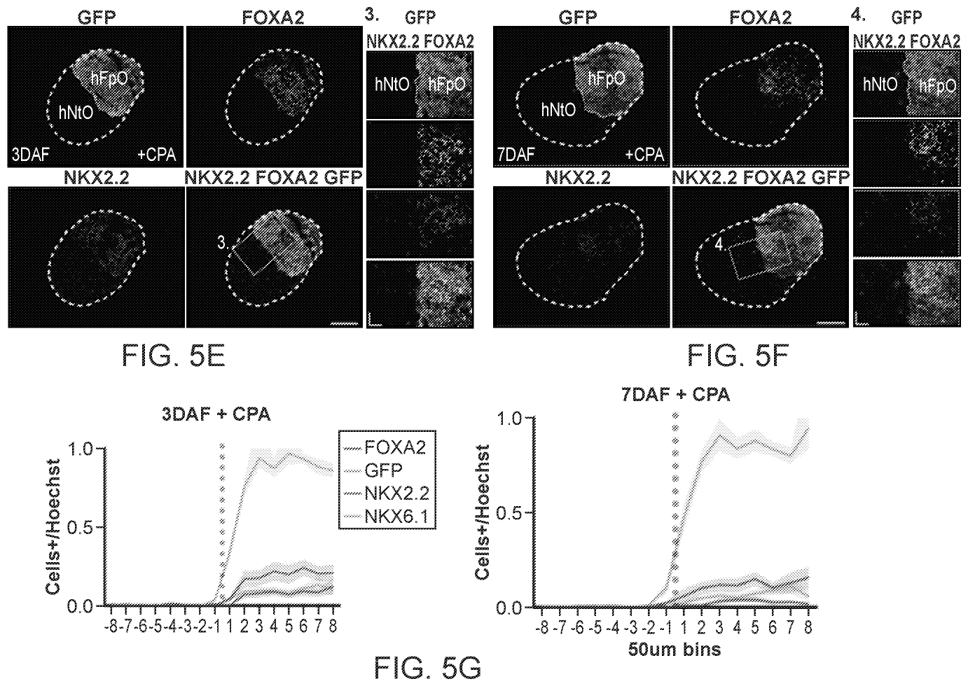

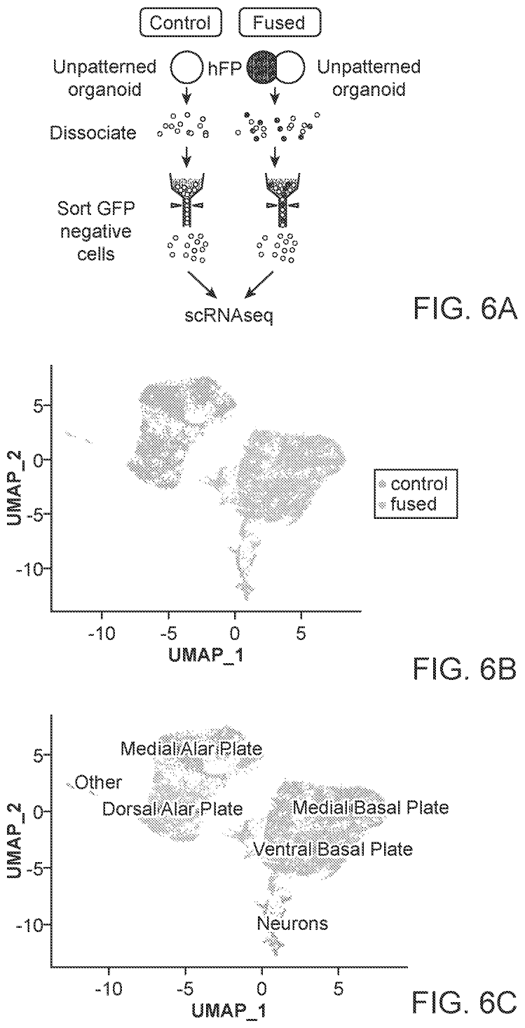

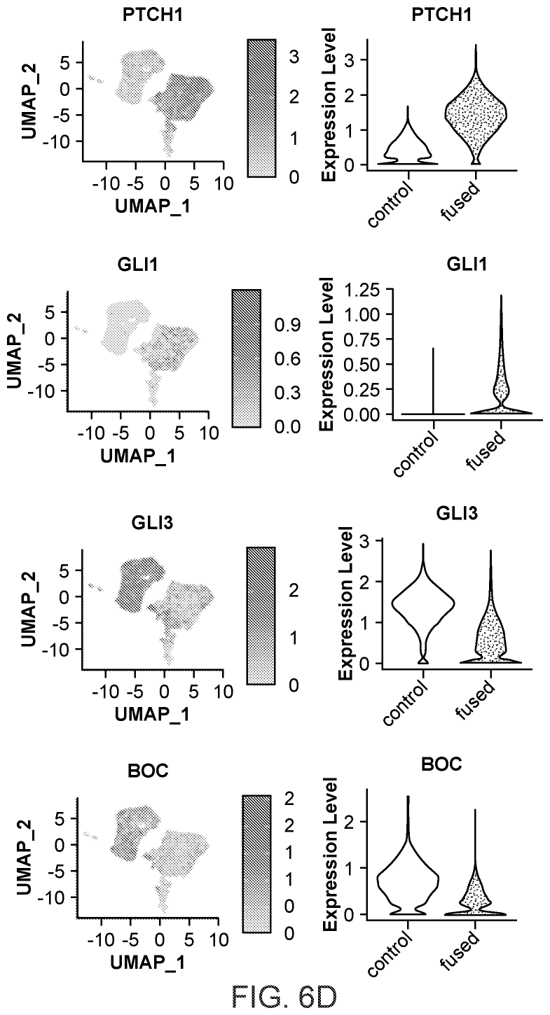

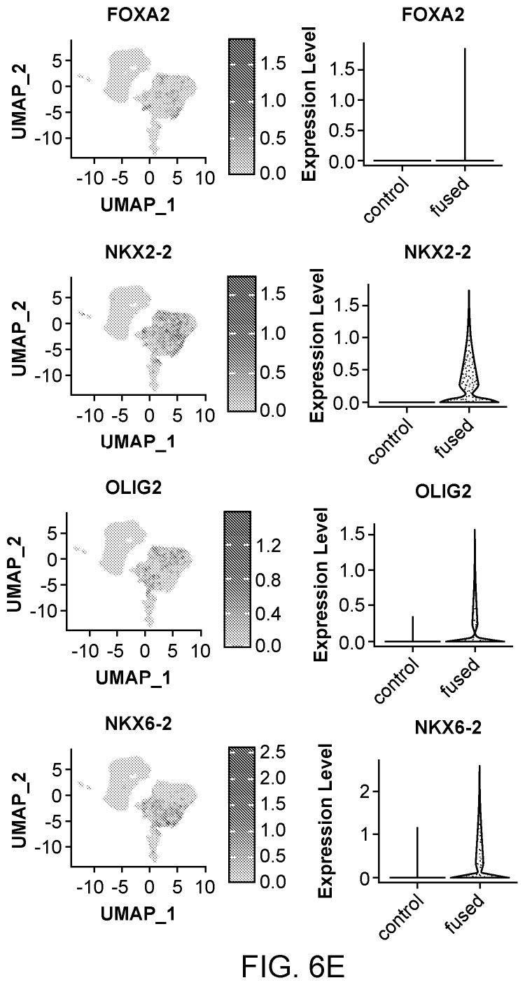

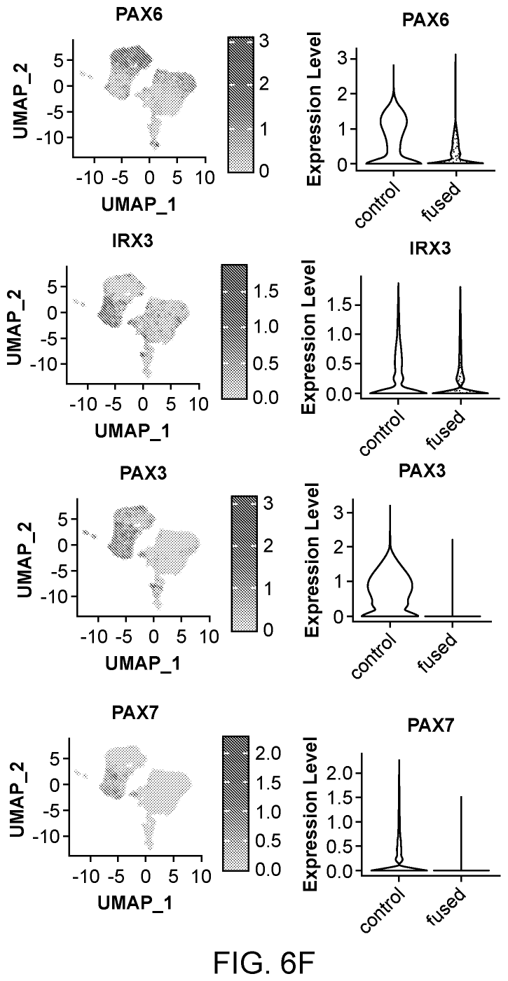

Generation of midline assembloids (hFpO-hNtO). To develop a model for the formation of the ventral spinal cord, hFpO was assembled with organoids that resembled the caudal neural tube (FIG. 1). Neural organoids were generated and exposed to the dual SMAD inhibition for 6 days and then transferred them to media containing EGF, FGF2, CHIR, and RA. Both hFpO (labeled with GFP) and hNtO were differentiated separately, dissociated to control for size, and then reaggregated in ultra-low round bottom plates. The following day, hFpO and hNtO were joined in the same well and allowed to fuse for 24 hours. At days 3 and day 7 to see if the hFpO can induce ventralization on the hNtO (FIG. 5). At day 3, ventralization was identified throughout the hNtO via immunofluorescence staining of the transcription factor NKX2.2 while the floor plate marker FOXA2 was retained to the GFP expressing region. By day 7, NKX2.2 expression was much more robust and FOXA2 expressing cells has been induced on the hNtO. This induction was proven to be SHH-dependent as treatment with the potent SHH antagonist cyclopamine abolished not only NKX2.2 induction but also all FOXA2 expression in hFpO (FIG. 5). To further our understanding of ventral induction via hFpO, single cell RNAseq was performed on cells patterned by the hFpO and compared to unfused hNtO (FIG. 6). Activation of SHH pathway genes in fused clusters which were absent in the control group were found. Furthermore, the activation of ventral neural progenitor markers and the repression of genes associated with dorsal neural tube development in this population was validated (FIG. 6). These experiments show that hiPSC cell-derived hFpO has induced robust ventralization in a SHH dependent manner on unpatterned tissue.

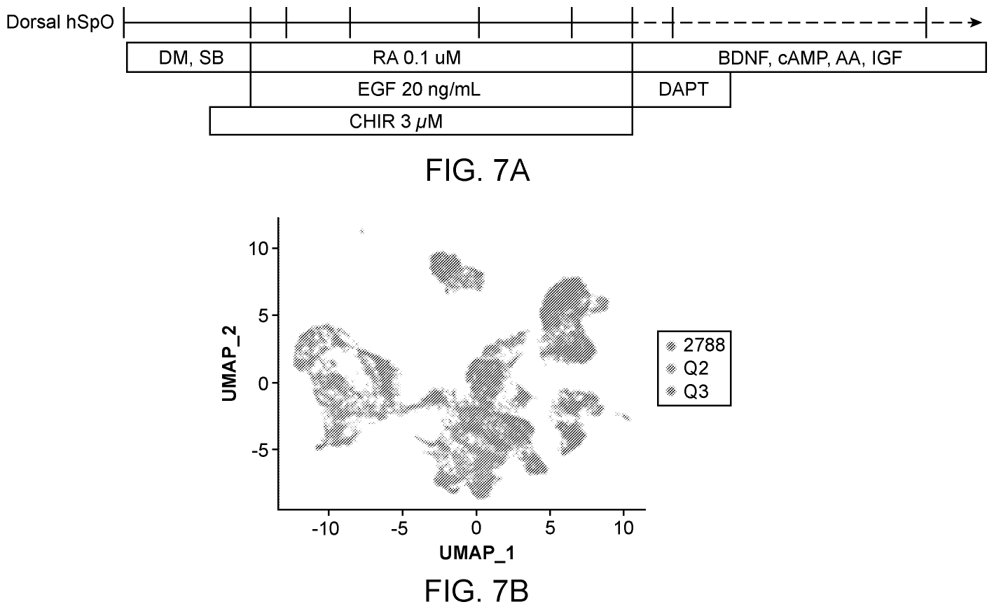

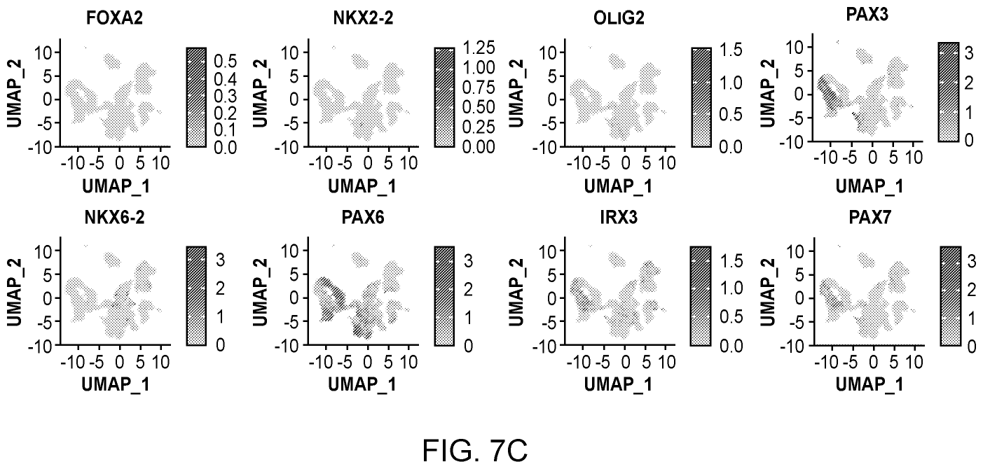

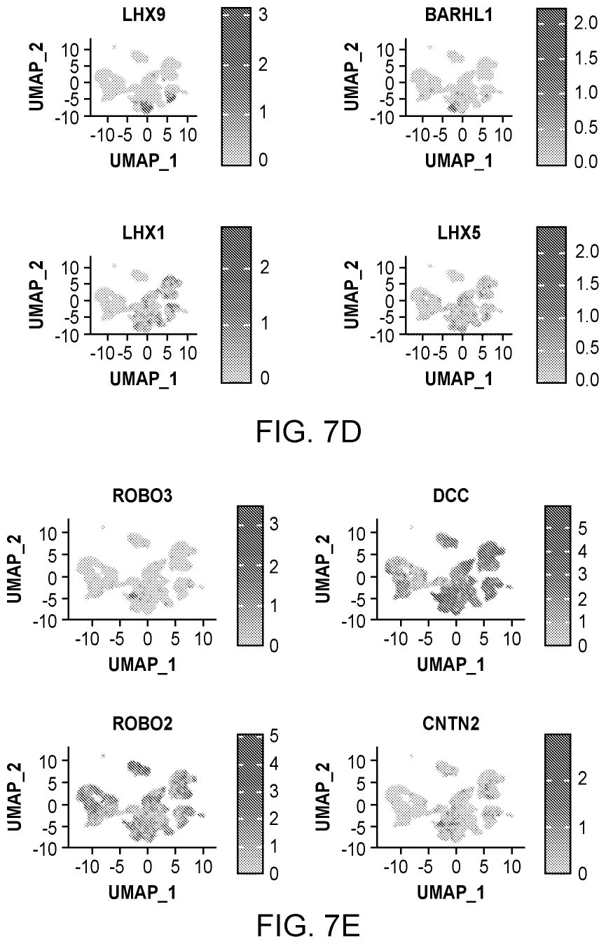

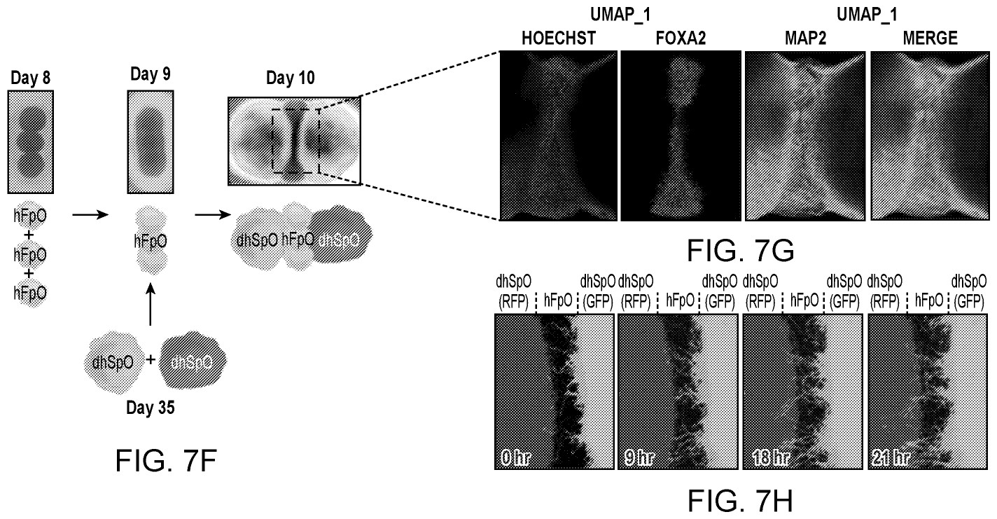

Generation of dhSpO and midline assembloids to study human midline crossing. To develop a model to study midline crossing in humans, using our hFpOs organoids that contain cell populations that are known to cross the floor plate in the developing spinal cord were generated. A spinal organoid differentiation protocol was performed originally described in (Anderson et al. 2020). To generate dorsal populations, molecules that are typically used to ventralize tissue such as SAG and FGF2 were excluded (FIG. 7). scRNAseq was performed on these organoids and found the expression of dorsal progenitor and neuron populations (FIG. 7). Additionally, these organoids excluded the expression of ventral markers such as FOXA2, NKX2.2, OLIG2 and NKX6.2. Cell populations of dorsal interneurons that are known to cross the midline in animal models were specifically identified in addition to the expression of hallmark commissural neuron genes (FIG. 7).

To generate midline assembloids to study the crossing of these axons, flanking dhSpO were fused to a linearly fused hFpO (FIG. 7). Several days after fusion, IHC revealed the restriction of FOXA2 expression to only the hFpO in the midline of the assembloid. Additionally, MAP2 positive projections were seen crossing through the hFpO and to the contralaterally positioned dhSpO (FIG. 7). The live extension and crossing of commissural neurons can be observed by generating dhSpO in a stem cell line that endogenously expresses GFP (FIG. 7). These experiments demonstrate the establishment of a platform to study the interaction of hFpO with dhSpO commissural axons in vitro.

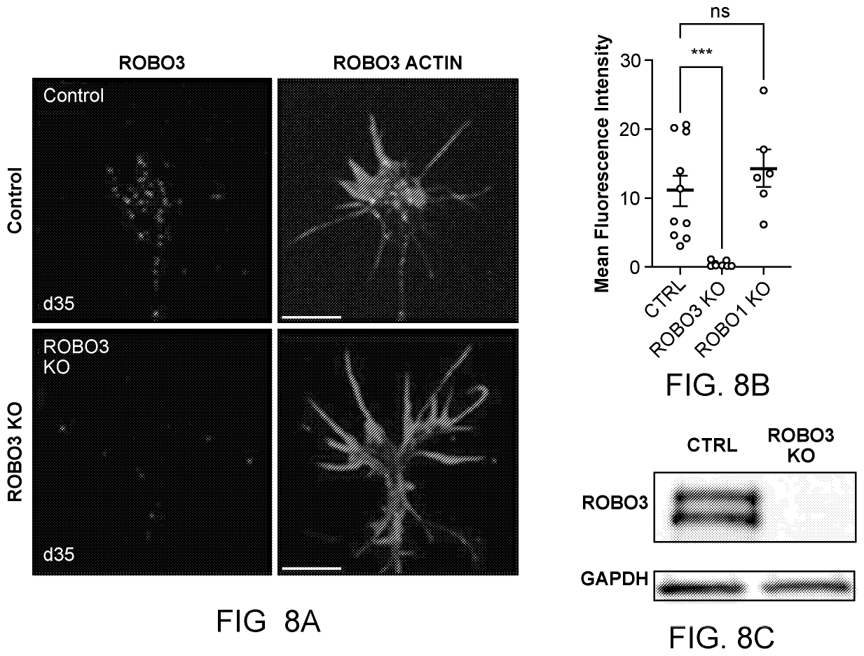

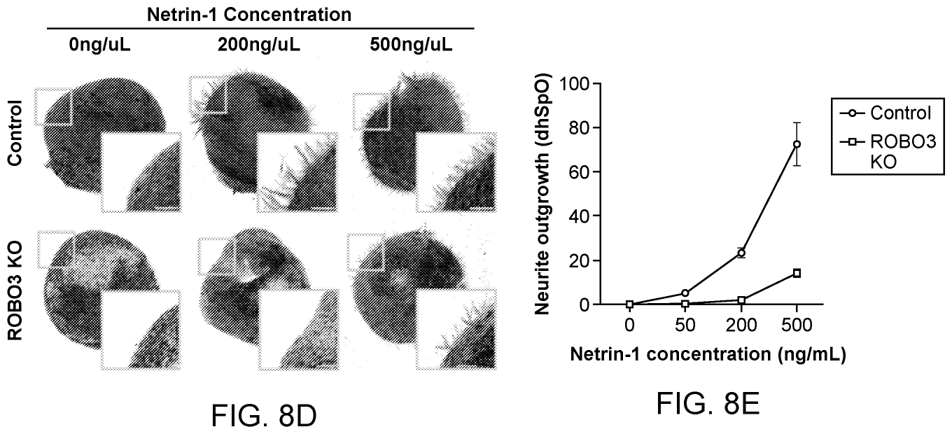

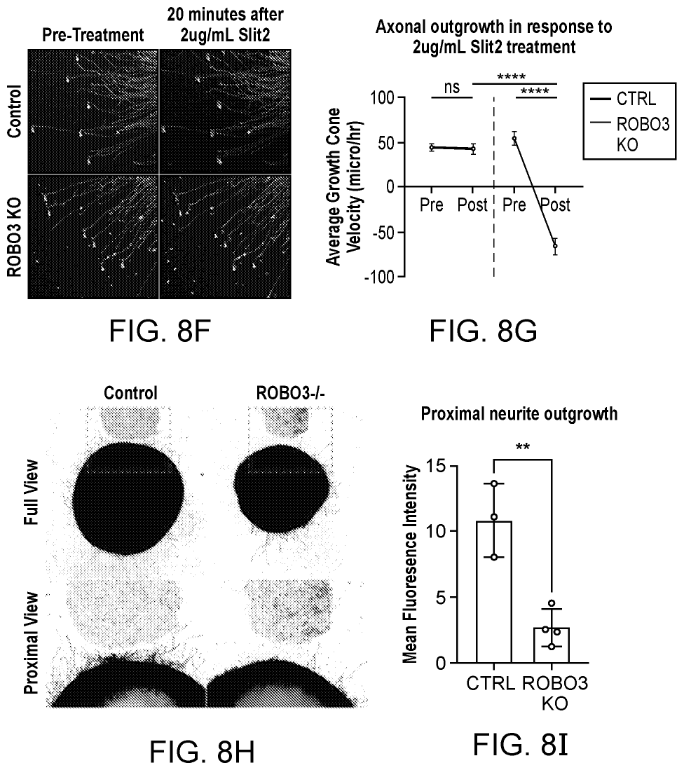

hFpO can stimulate axon extension in a ROBO3 dependent manner. To implement this model to study questions in axon guidance, classic assays were performed to test the functionality of hiPSC cell-derived floor plate on growth cone signal transduction at the midline. Additionally, known regulators of axon guidance (ROBO3) were sought to confirm hFpO functionality and implication of axon guidance defects in associated diseases (FIG. 8). In these assays human spinal organoids were generated to test whether the neurons generated in these models are chemoattracted to factors secreted by the floor plate and whether ROBO3 KO will perturb any elicited responses. First, responsivity of dorsal interneuron axon extension responsivity was tested to purified FP-secreted proteins. It was found that netrin has the ability stimulate axon extension with increasing doses while netrin-induced outgrowth was diminished in ROBO3 KO dhSpOs. Furthermore, it was found that wildtype dorsal interneurons are largely insensitive to the repulsive axon guidance cue SLIT-2 while ROBO3 KO neurites show increased sensitivity and chemorepulsion. Finally, day-30 hSpO were embedded at 150 microns from a day-8 hFpO. Following matrix polymerization and overnight incubation, robust neurite attraction to the hFpO was found (FIG. 7). A dramatic reduction in the number of proximal projections was observed in the ROBO3 KO dhSpOs, suggesting FP-induced neurite extension is dependent on ROBO3 expression. These experiments show that the hFpO secretes factors that are specific to axon guidance at the human midline and that ROBO3 plays a crucial role in this process and is conserved across species.

These data demonstrate the first 3D human model of the human ventral midline generated by in vitro assembly starting from human pluripotent stem cells. Several applications can be carried out for this system. hFpO implication in neural patterning in the spinal cord shows great promise in modeling diseases that cause defective neural tube formation (holoprosencephaly) while the ability for hFpO to modulate the guidance of axons can be key for studying human neurodevelopmental disorders that have defects in midline crossing, for example schizophrenia, autism, tuberous sclerosis, etc. A major advantage this model has over other organoid model systems is the extremely short period it takes to generate these assembloids allowing for highly scalable screens for drugs and genes that are relevant for neural tube formation and axon guidance at the midline.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method for producing substantially pure functional human floor plate cells in vitro, the method comprising:
culturing in suspension human induced pluripotent cells in the presence of an effective dose of one or more SMAD inhibitors;
adding to the suspension culture after one day and continuing for 6 days, an effective dose of an SHH pathway agonist, optionally in the presence of an effective dose of epidermal growth factor, fibroblast growth factor and/or retinoic acid;
supplementing the medium with an effective dose of a GSK-3 inhibitor after day 2 and continuing for 6 days;
to generate organoids comprising a substantially pure population of human floor plate cells (hFpO).

2. The method of claim 1, wherein the human floor plate cells express the marker FOXA2.

3. The method of claim 1 or claim 2, wherein the human floor plate cells induce ventralization in neural cells.

4. The method of any of claims 1-3, wherein the human floor plate cells express transcription factors, morphogens and axon guidance molecules.

5. The method of any of claims 1-4, wherein the human floor plate cells express sonic hedgehog (SHH).

6. The method of any of claims 1-5, wherein the human floor plate cells attract or repel neurons from a spinal cord organoid in a matrix culture.

7. The method of any of claims 1-6, wherein the human floor plate cells comprise at least one allele associated with a neurologic disorder.

8. The method of any of claims 1-7, wherein the SMAD inhibitors comprise a dose of dorsomorphin (DM) or LDN 193189, and SB-431542 effective to induce pluripotent stem cells to a neural fate.

9. The method of any of claims 1-8, wherein the suspension culture is feeder layer free.

10. A method for producing substantially pure functional human roof plate cells in vitro, the method comprising:
culturing in suspension human induced pluripotent cells in the presence of an effective dose of one or more SMAD inhibitors;
adding to the suspension culture after one day and continuing for 6 days, an effective dose of an BMP pathway agonist, optionally in the presence of an effective dose of epidermal growth factor, fibroblast growth factor and/or retinoic acid;
supplementing the medium with an effective dose of a GSK-3 inhibitor after day 2 and continuing for 6 days;
to generate organoids comprising a substantially pure population of human roof plate cells.

11. The method of claim 10, wherein the SMAD inhibitors comprise a dose of dorsomorphin (DM) or LDN 193189, and SB-431542 effective to induce pluripotent stem cells to a neural fate.

12. The method of claim 10 or 11, wherein the GSK-3 inhibitor is CHIR 99021.

13. A method for producing a human neural tube organoid (hNtO), the method comprising:
(a) culturing in suspension human induced pluripotent cells in the presence of an effective dose of one or more SMAD inhibitors for 4 to 6 days;
(b) transferring the cells of step (a) to a suspension culture comprising an effective dose of epidermal growth factor, fibroblast growth factor, a GSK-3 inhibitor, and retinoic acid;
(c) culturing in suspension the cells of step (b) for 1 to 5 days;
to generate organoids comprising human neural tube cells.

14. The method of claim 13, wherein the SMAD inhibitors comprise a dose of dorsomorphin (DM) or LDN 193189, and SB-431542 effective to induce pluripotent stem cells to a neural fate.

15. The method of claim 13 or 14, wherein the GSK-3 inhibitor is CHIR 99021.

16. A method for producing a hFpO-hNtO midline assembloid, the method comprising:
(a) culturing in suspension human induced pluripotent cells in the presence of an effective dose of one or more SMAD inhibitors for 4 to 6 days;
(b) transferring the cells of step (a) to a suspension culture comprising an effective dose of epidermal growth factor, fibroblast growth factor, a GSK-3 inhibitor, and retinoic acid;
(c) culturing in suspension the cells of step (b) for 1 to 5 days thereby producing a hNtO; and
(d) culturing in suspension the hFpO of claim 1 and the hNTO under conditions permissive for cell fusion in a neural medium, such that the hFpO induces ventralization of the hNtO in the hFpO-hNtO midline assembloid.

17. The method of claim 16, wherein the ventralization of the hNTO in the hFpO-hNtO midline assembloid results in an increase in the expression of one or more genes selected from the group consisting of: FOXA2, NKX2-2, OLIG2 and NKX6-2.

18. The method of claim 16 or 17, wherein the ventralization of the hNTO in the hFpO-hNtO midline assembloid results in a decrease in the expression of one or more genes selected from the group consisting of: PAX6, IRX3, PAX3, and PAX7.

19. The method of any of claims 16-18, wherein the ventralization of the hNTO in the hFpO-hNtO midline assembloid results in increase expression of PTCH1, increase expression of GLI1, decreased expression of GLI3, decrease expression of BOC or any combination thereof.

20. The method of any of claims 16-19, wherein the ventralization of the hNTO in the hFpO-hNtO midline assembloid is sonic hedgehog dependent.

21. A method of producing a dorsal human spinal organoid (dhSpO)-hFpO-dhSpO midline assembloid, the method comprising:
(a) culturing in suspension human induced pluripotent cells in the presence of an effective dose of one or more SMAD inhibitors for 4 to 6 days;
(b) transferring the cells of step (a) to a suspension culture comprising an effective dose of epidermal growth factor, a GSK-3 inhibitor, and retinoic acid;
(c) culturing in suspension the cells of step (b) for 10-15 days;
(d) culturing in suspension the cells of step (c) in neural media comprising at least one compound selected from the group consisting of brain-derived neurotrophic factor (BDNF), Insulin-like growth factor (IGF), L-Ascorbic Acid 2-phosphate Trisodium Salt (AA), N6, 2′-O-Dibutyryladenosine 3′, 5′-cyclic monophosphate sodium salt (cAMP), thereby generating a dhSpO and
(d) culturing the hFpO of claim 1 between two dhSpO under conditions permissive for cell fusion in a neural medium, such that axons project from one dhSpO through the hFpO to the other dhSpO.

22. The method of claim 21, wherein the axons express MAP2.

23. The method of claim 21 or 22, wherein only the hFpO of the dhSpO-hFpO-dhSpO midline assembloid expresses FOXA2.

24. The method of any of claims 21-23, wherein the dhSPO of the dhSpO-hFpO-dhSpO midline assembloid do not express ventral marker genes.

25. The method of 24, wherein the ventral marker genes are selected from the group consisting of FOXA2, NKX2.2, OLIG2, NKX6.2, and any combination thereof.

26. A method determining the effect of a candidate agent on human spatial patterning and neural connectivity, the method comprising: contacting the candidate agent with one or a panel of organoids produced by the methods of any of claims 1-15, or an assembloid of claim 16-25, or a population of cells isolated therefrom; and determining the effect of the agent on morphologic, genetic or functional parameters.

27. The method of claim 26, wherein the functional parameter is ventralization of the organoids or assembloids, axon growth, or axon guidance.
